(19) United States
(12) Patent Application Publication
(10) Pub. No.: US 2025/0161407 A1
(43) Pub. Date: May 22, 2025

(54) Medical application and pharmaceutical composition of UbV.E4B protein

(71) Applicant: ZHEJIANG UNIVERSITY, Hangzhou (CN)

(72) Inventor: Xuhua WANG, Hangzhou (CN)

(21) Appl. No.: 18/963,963

(22) Filed: Nov. 29, 2024

Related U.S. Application Data
(63) Continuation of application No. PCT/CN2022/142349, filed on Dec. 27, 2022.

(30) Foreign Application Priority Data
Jun. 1, 2022 (CN) 2022106216521

(51) Int. Cl. A61K 38/17; A61P 25/00; C07K 14/47

(52) U.S. Cl. CPC A61K 38/1709; A61P 25/00; C07K 14/47

(57) ABSTRACT

Provided is use of UbV.E4B protein in the preparation of a medicament for promoting central nervous regeneration. The UbV.E4B protein promotes the axonal regeneration of a central nervous system by selectively inhibiting a Ube4b ubiquitination factor. Provided is use of the Ube4b ubiquitination factor as a screening target in screening a Incdicament for promoting the axonal regeneration of the central nervous system. Further provided is a pharmaceutical composition for promoting the central nervous axonal regeneration.

### REFERENCE TO SEQUENCE LISTING

The instant application contains a Sequence Listing in XML format as a file named “ZDPT240010US-seq.xml”, created on Nov. 28, 2024, of 2377 bytes in size, and which is hereby incorporated by reference in its entirety.

### TECHNICAL FIELD

The invention relates to the field of biological pharmacy, in particular to the use of UbV.E4B protein in preparing medicaments for promoting central nerve regeneration, application of Ube4b ubiquitination factor as a screening target in screening a medicament for promoting central nervous system axon regeneration and a pharmaceutical composition related to the UbV.E4B protein or the Ube4b ubiquitination factor.

### BACKGROUND

The nervous system has a major role in regulating physiological functions in the body and is divided into the Central Nervous System (CNS) and the Peripheral Nervous System (PNS). Most axons of the central nervous system have limited ability to regenerate after injury, which is a major obstacle to functional recovery after injury. Therefore, the research on the potential molecular mechanism for determining nerve regeneration plays an important role in nerve repair and functional recovery after injury.

The intrinsic regenerative capacity of corticospinal tract (CST) axons is limited, preventing functional recovery after cortical stroke. One promising strategy to restore dysfunction after cortical stroke is to utilize neonatal corticospinal tract (CST) axons to re-innervate the spinal cord. Unfortunately, CST axons in the adult central nervous system are inherently difficult to regenerate after cortical stroke. Understanding the fundamental biological processe of intrinsic inhibition of axon regeneration within neurons is a prerequisite for the development of interventions in stroke or Central Nervous System (CNS) injury diseases. Over the past decade, several key regulators have been investigated to affect the intrinsic regenerative capacity of nerve cells. Modulation of other signaling factors downstream of PI3K, such as S6K1 and GSK-3, also promotes significant axon regeneration. SOCS3 is a negative regulator of the JAK/STAT3 pathway, and knocking out SOCS3 induces axon regeneration. Research has shown that the rapid increase in DLK expression after Retinal Ganglion Cell (RGC) damage can promote the expression of regenerative genes. Modifications to neuronal transcription programs such as knockout of KLF4, overexpression of p300 in RGC, overexpression of KLF7 or RARB in CST, all promote axon regeneration after injury. Gene engineering mediated methods such as SOX11, Lin28 can block the expression of nerve regeneration inhibitory genes, and have the effect of obviously promoting central nerve regeneration. These studies indicate that increasing the activity of mammalian rapamycin (mTOR) can significantly enhance the axon regeneration capacity of injured neurons. At the same time, p53 pathway has also been suggested to modulate the intrinsic regenerative capacity of central nervous system neurons. However, the nerve regeneration promoting methods based on the currently known pathways are not ideal, and there is still a need to develop other routes for promoting nerve regeneration, particularly methods or medicaments capable of promoting central axon regeneration.

On the other hand, the screening methods of existing medicaments capable of promoting nerve regeneration are mainly animal models, effective molecular screening model is lacked, the development speed of new medicaments is greatly inhibited, and is an industry problem to be solved urgently.

### SUMMARY

The ubiquitin pathway of organism's protein quality control system is responsible for maintaining a balance of the cellular internal environment by stabilizing proteins and degrading misfolded proteins. Ubiquitination is an important post-translational modification of proteins and is one of the major pathways for protein degradation in the body. After axonal injury, neurons need to rapidly restore cellular balance and synthesize abundant proteins for axon regeneration, and thus the ubiquitination pathway is considered to play an important role in this process. But knowledge of how and to what extent they regulate central nerve axon regeneration remains sporadic. Since the ubiquitin pathway is implicated in regulating axon regeneration through p53 axon, the inventors speculate that ubiquitin molecules that regulate p53 degradation may play an important role in controlling central nervous axon regeneration. To test this hypothesis, the inventors screened a series of genes that modulate the ubiquitin pathway that degrades p53 protein and identified ubiquitination factor E4B (Ube4b), an E3 and E4 ubiquitin ligase, as inhibitors of central nerve axon regeneration. Surprisingly, the inventors found that Ube4b also plays a key role in regulating mTOR, the major control pathway for axon regeneration in the central nervous system. More importantly, the inventors achieved CST sprouting and CST-dependent behavior recovery by overexpressing ubiquitin variant UbV.E4B to specifically inhibit the activity of Ube4b in the rat cortical stroke model, thereby completing the object of the present invention. Specifically, the invention provides the use of UbV.E4B in medicaments for promoting central nervous system axon regeneration, wherein the UbV.E4B promotes central nervous system axon regeneration by selectively inhibiting Ube4b ubiquitination factor protein.

Based on the same principle, the invention also provides the use of nucleic acid sequence for encoding the UbV.E4B protein in preparing medicaments for promoting central nerve regeneration characterized in that the protein obtained by expressing the UbV.E4B gene selectively inhibits Ube4b ubiquitination factor, thereby promoting central nervous system axon regeneration.

In a preferred embodiment of the invention, the central nerve axon regeneration is optic nerve and/or brain, spinal nerve axon regeneration.

The invention provides the use of Ube4b protein as a target substance in medicament screening for promoting central nerve axon regeneration.

In a preferred embodiment of the invention, the medicament for promoting central nerve regeneration is an inhibitor of Ube4b protein.

In a preferred embodiment of present invention, the medicament for promoting central nerve regeneration is a protein, an antibody, a nucleic acid medicament, a small molecule medicament.

In a preferred embodiment of the invention, the medicament screening for promoting central nerve regeneration is screening for a substance capable of allosterically inactivating Ube4b ubiquitination factor protein,
or screening for a substance capable of competitively occupying the natural action site of the Ube4b ubiquitination factor protein,
or screening for a substance capable of inhibiting the biosynthesis of the Ube4b ubiquitination factor protein in vivo.

The invention also provides a pharmaceutical composition for promoting central nerve regeneration comprising a Ube4b ubiquitination factor inhibitor and a pharmaceutically acceptable carrier.

The pharmaceutical composition of the invention, further compromising a related inhibitor of PTEN gene, and/or a related inhibitor of MDM2 gene. The pharmaceutical composition of the invention further compromising a MDM2-p53 inhibitor, as a representative of MDM2-p53, the following APG-115 can be given. The inhibitor has synergistic effect with Ube4b ubiquitination factor inhibitor and can bring better nerve regeneration curative effect.

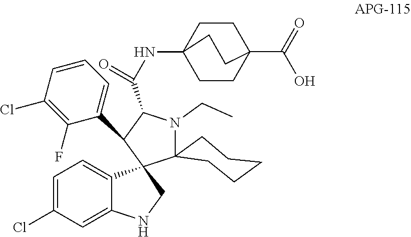 [SMILES: CCN1[C@@H](C(=O)NC23CCC(C(=O)O)(CC2)CC3)[C@H](c2cccc(Cl)c2F)[C@]2(CNc3cc(Cl)ccc32)C12CCCCC2]

The pharmaceutical composition of the invention preferably comprises a medicament that has similar effect as APG-115.

In a preferred embodiment of the invention, the Ube4b ubiquitination factor protein inhibitor is the UbV.E4B protein or a vector comprising nucleic acid sequence encoding the UbV.E4B protein. It may be formulated for administration to the brain, spinal cord, or optic nerve.

The pharmaceutical composition of the invention formulated for administration by an intracerebroventricular, intranasal, intracranial, intraventricular, intracerebellar, or intrathecal route.

More specifically, the invention also provides a modified UbV.E4B protein linked to a membrane-penetrating peptide. More specifically, the invention provides a gene sequence encoding the modified UbV.E4B protein of claim 13, as shown in SEQ ID NO: 1.

Compared with the prior art, the invention has the following remarkable characteristics.
1. In the existing research reports, the key regulation mechanism for simultaneously regulating the two main pathways is rarely known in the process of controlling central nervous system axon regeneration, and the invention discloses the key mechanism for simultaneously regulating the two main central nerve axon regeneration control pathways of p53 and mTOR, thereby providing a possible cure pathway for recovering CST dependent function after cortical stroke;
2. The accelerated nerve regeneration related to the Ube4b gene or protein inhibition does not affect the survival rate of neurons, and has good safety;
3. The Ube4b gene or protein function inhibition can be quickly and simply used for establishing a medicament screening model, can be used for screening protein, antibody, nucleic acid and small molecule medicaments which are effective to the Ube4b gene or protein inhibition, can quickly establish a molecular level model, and greatly improves the research and development speed of this kind of medicaments compared with animal model;
4. The invention provides a method for promoting nerve regeneration by synergistically using a strategy for inhibiting the function of Ube4b gene or protein and a strategy for inhibiting the transcription or expression associated with PTEN, which has a more excellent effect; meanwhile, the blocking of the MDM2 gene has a synergistic effect with the blocking of Ube4b, and the blocking of MDM2 gene and Ube4b gene has more excellent effect on nerve regeneration.
5. Gene therapy and protein therapy of UbV.E4B have the potential for clinical transformation.

### BRIEF DESCRIPTION OF FIGURES

FIG. 1A-1G is a graph showing that UbV.E4B treatment promotes regeneration of CST axons in the spinal cord and improves skilled motor function after unilateral cortical stroke;

FIG. 2A-2C is a graph showing that Ube4b is a key regulator capable of regulating central nerve axon regeneration;

FIG. 3A-3K is a graph showing that deletion of Ube4b promotes optic nerve regeneration and upregulates p53 and mTOR;

FIG. 4A-4D is a graph showing that the knockout of Ube4b gene modulates axon regeneration by two-pathway mediation;

FIG. 5A-5F is a graph showing that the double gene knockout of Ube4b and PTEN further promotes optic nerve regeneration;

FIG. 6A-6F is a graph showing ablation of regenerative CSNs defeating recovery of proficient motor abilities;

FIG. 7A-7B is a graph showing the effect of Ube4b knockout on RGC survival after injury;

FIG. 8A-8B is a graph showing that knockout of p53 or Klhl22 has no significant effect on survival of RGCs after injury;

FIG. 9A-9C is a graph showing Ube4b knockout up-regulates p53 and mTOR in the brain;

FIG. 10A-10D is a graph showing that Ube4b knockout promotes CST axonal sprouting in the spinal cord after unilateral cortical stroke;

FIG. 11A-11D is a graph showing that overexpression of UbV.E4B promotes optic nerve regeneration;

FIG. 12A-12B is a graph showing overexpression of HA-UbV.E4B in the sensory motor cortex;

FIG. 13A-13D is a graph showing that UbV.E4B treatment has no significant effect on the reach step;

FIG. 14A-14E is a graph showing that the gene knockout of MDM2 can promote optic nerve regeneration; the double gene knockout of Ube4b and MDM2 can further promote optic nerve regeneration;

FIG. 15A-15B shows frequency table of E. coli codon usage and sequence alignment before and after codon optimization of UbV.E4B gene;

FIG. 16A-16E shows the method and procedure for pET-SUMO-UbV.E4B vector construction and expression identification;

FIG. 17A-17B shows Tricine-SDS-PAGE electrophoresis results after in vitro cleavage of recombinant proteins;

FIG. 18A-18D shows the results of neurite regeneration of in vitro cultured neurons promoted by entry of rUbV.E4B protein into cells;

FIG. 19A-19E shows the results of promoting optic nerve regeneration by rUbV.E4B protein.

### DETAILED DESCRIPTION

. The present invention will be described in further detail with reference to examples and drawings, but the embodiments of the present invention are not limited thereto. Description of the terms

According to the general biological principle, genes encode proteins, and the proteins are biosynthesized by transcription and translation of the genes, so that when the Ube4b is generally called, the Ube4b gene can be represented, and the Ube4b ubiquitination factor which is the protein expressed by the Ube4b gene can be represented. As a ubiquitin variant corresponding to Ube4b ubiquitination factor, the sequence of UbV.E4B gene is known (refer to Gabrielsen, M. et al. A General Strategy for Discovery of Inhibitors and Activators of RING and U-box E3 Ligases with Ubiquitin variants. Mol Cell 68, 456-470E 410, doi:10.1016/j.molcel.2017.09.027 (2017)). Based on the same principle as above, when called UbV.E4B, it may represent either the UbV.E4B gene or the protein expressed based on the UbV.E4B gene sequence, the UbV.E4B protein, the expression means only the protein expressed based on the UbV.E4B gene sequence.

The use of the terms “a” and “an” and “the” and similar referents in the context of describing the invention as presently claimed (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

The use of the term “about” is intended to describe values above or below the stated value within a range of about +/−10%; in other embodiments, these values may be within a range of values above or below the specified value within a range of about +/−5%; in other embodiments, these values are within a range of values above or below the stated values within a range of about +/−2%; in other embodiments, these values are within a range of values above or below the stated values within a range of about +/−1%. The foregoing scope is intended to be clear from the context and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “carrier” refers to an organic or inorganic, natural or synthetic inactive ingredient (or one or more active ingredients in combination therewith) in the formulation.

In some expressions, the carrier or excipient may be an inert substance added to the pharmaceutical composition to further facilitate administration of the compound, and/or does not cause significant irritation to the organism and does not abrogate the biological activity and properties of the administered compound. The carrier may be composed of, for example, materials that are considered safe and effective and that can be administered to an individual without causing undesirable biological side effects or undesirable interactions. The carrier may be, for example, any component present in a pharmaceutical formulation other than the active ingredient or ingredients. The term “carrier” includes, but is not limited to, diluents, binders, lubricants, disintegrants, fillers, and coating compositions. Vectors also include means by which biologically plasmids, bacteriophages, adenoviruses, etc. can carry genes for expression of the desired proteins.

The term “effective amount” in connection with a compound or composition refers to an amount of the compound that is non-toxic but sufficient to provide a desired or reference result. The term “therapeutically effective amount” of a compound refers to an amount of the compound that is non-toxic but sufficient to provide the desired or reference therapeutic result. For example, an effective amount can refer to a dose sufficient to reduce or inhibit the disorder, disease, or condition being treated, or to otherwise provide a desired pharmacological and/or physiological effect. The precise dosage will vary depending on a variety of factors such as the subject-related variables (e.g., age, immune system health, etc.), the severity of the disease or disorder being treated, and the route of administration and pharmacokinetics of the agent being administered. As discussed elsewhere herein, and as is known, the exact amount required will vary from subject to subject, depending on the species, age, and general condition of the subject, the severity of the disease being treated, the particular compound used, its mode of administration, and the like. Therefore, it is not possible to specify an exact “effective amount”. However, the appropriate effective amount can be determined by one of ordinary skill in the art using only routine experimentation.

The terms “pharmaceutical”, “pharmaceutically acceptable” refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio. The term “pharmaceutical carrier” refers to all components of a pharmaceutical formulation that facilitate delivery of the composition in vivo. pharmaceutical carriers include, but are not limited to, diluents, preservatives, binders, lubricants, disintegrants, swelling agents, fillers, stabilizers, and combinations thereof.

The terms “inhibit,” “suppress,” “reduce,” “interfere with,” and/or “reduce” (and similar terms) generally refer to an action that directly or indirectly reduces a function, activity, level, concentration, behavior, etc., relative to the natural, intended, or average, or relative to the present state. It will be appreciated that this is typically related to some standard or expected value, in other words it is relative, but it does not always require reference to a standard or relative value. For example, an agent that inhibits, suppresses, reduces or otherwise reduces or interferes with bone loss may stop or slow osteoblast apoptosis or osteoclast activity. This may be a complete inhibition, suppression, reduction, interference and/or reduction of function, activity, level, concentration, behavior, etc. Inhibition, suppression, reduction, interference and/or reduction may be compared to a control or standard level. The inhibition, suppression, reduction, interference, and/or reduction may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The terms “increase,” “enhance,” “stimulate,” “promote,” and/or “induce” (and similar terms) generally refer to an action that improves or increases function, activity, level, concentration, behavior, etc., either directly or indirectly, relative to natural, expected, or average, or relative to the present condition. It will be appreciated that this is typically related to a certain standard or expected value, in other words it is relative, but it does not always require reference to a standard or relative value. For example, in the context of osteoblast differentiation, a substance that increases, stimulates, promotes, induces or enhances bone formation may induce the production and/or secretion of osteogenic molecules such as alkaline phosphatase, osteocalcin, osteopontin, osteonectin, bone sialoprotein, and collagen 1a 1. The increase, enhancement, stimulation, promotion and/or induction can be compared to a control or standard level. The increase, enhancement, stimulation, promotion and/or induction may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 320%, 325%, 340%, 350%, 360%, 375%, 380%, 400%, 425%, 450%, 475%, 500%, 550%, 600%, 650%, 700%, 750%, 800%, 850%, 900%, 103%, 104%, 105%, 106%, or 107%.

The terms “treat” and “treatment” refer to the medical management of a subject with the intent to cure, ameliorate, stabilize, or prevent a disease, pathological condition, or disorder. The term includes active treatment, i.e., treatment directed specifically to ameliorating a disease, pathological condition, or disorder, and also includes causal treatment, i.e., treatment directed to eliminating the cause of the associated disease, pathological condition, or disorder. Moreover, the term also includes palliative treatments, i.e., treatments designed to alleviate symptoms rather than cure a disease, pathological condition, or disorder; prophylactic treatment, i.e., treatment directed to minimizing or partially or completely inhibiting the development of an associated disease, pathological condition, or disorder; and supportive treatment, i.e. treatment for supplementing another specific treatment aimed at improving the relevant disease, pathological condition or disorder. It is to be understood that treatment, while intended to cure, ameliorate, stabilize or prevent a disease, pathological condition or disorder, does not actually result in a cure, amelioration, stabilization or prevention. The effect of the treatment can be measured or assessed as described herein and as known in the art as appropriate for the disease, pathological condition or disorder involved. Such measurements and assessments can be made in a qualitative and/or quantitative manner. Thus, for example, the identity or characteristic of a disease, pathological condition or disorder and/or the symptoms of a disease, pathological condition or disorder can be reduced to any effect or to any amount.

The terms “apply” “give” and “administer” refer to bringing a substance, material, or product into contact with the body of a subject. For example, applying a substance, material, or product includes contacting the skin of a subject and injecting or implanting the substance, material, or product into the subject.

The terms “sufficient” and “effective,” used interchangeably, refer to an amount (e.g., mass, volume, dose, concentration, and/or time period) necessary to achieve one or more desired results.

In the context of genes and gene products, the term “expression” refers to the process by which information from a gene is used to synthesize a functional gene product. Expression can be measured in a variety of ways, including, for example, by measuring the level of one or more products of gene expression.

The term “protein level” refers to the level (e.g., amount, concentration) of one or more reference proteins. The term “transcript level” refers to the level (e.g., amount, concentration) of one or more reference transcripts.

The terms “pathway” and “signaling pathway” refer to a group of molecules in a cell that work together to control one or more cellular functions, such as cell division or cell death. After the first molecule in the pathway receives the signal, it activates another molecule. This process is repeated until the last molecule is activated and performs a cellular function. Abnormal activation of signaling pathways can lead to cancer, and medicaments are being developed to block these pathways. These medicaments may help to prevent cancer cell growth and kill cancer cells. In the context of a signaling pathway, the term “protein level” refers to the level of one or more proteins that are part of the signaling pathway. In the context of a signaling pathway, the term “transcript level” refers to the transcript level of one or more genes that encode a protein or regulatory element that is part of the signaling pathway. In the context of a signaling pathway, the term “activity” refers to the level of activation of the signaling pathway.

As used herein, the terms “modulate” and “regulate” refer to the ability of a compound to alter activity in some measurable manner, as compared to an appropriate control. As a result of the presence of the compounds in the assay, the activity may be increased or decreased compared to a control in the absence of these compounds. Preferably, the increase in activity is at least 25%, more preferably at least 50%, most preferably at least 100% compared to the level of activity in the absence of the compound. Similarly, the reduction in activity is preferably at least 25%, more preferably at least 50%, most preferably at least 100% compared to the level of activity in the absence of the compound. Compounds that increase known activity are “agonists”. Compounds that reduce or prevent a known activity are “antagonists”.

As used herein, the terms “provide” and “administer” refer to any means of adding a compound or molecule to something known in the art. Examples provided may include the use of pipettes, pipettors, syringes, needles, tubes, guns, and the like. This may be manual or automatic. It may include transfection by any means or any other means of providing nucleic acid to a culture dish, cell, tissue, cell-free system, and may be performed in vitro or in vivo.

The meaning of terms is partially abbreviated in the invention.
RGC: retinal ganglion cells
CST: corticospinal tract
ONC: optic nerve clamp
PTEN: phosphoesterases and tensin homologues
CTB: cholera toxin B subunit
AAV: adeno-associated virus
MDM2: mouse double minute gene 2
SUMO: ubiquitin-like protein modifying molecule
HA: hemagglutinin tag
PLAP: placenta alkaline phosphatase
HAUSP: Herpesvirus-associated ubiquitin-specific protease
Pirh2: an E3 ubiquitin ligase regulated by p53
COP1: an E3 ubiquitin ligase
RBPMS: RNA binding protein participating in processing of mRNA
Cre: cyclization recombinase
Klhl22: Kelch protein family members
pS6: phosphorylated S6 ribosomal protein
Camk2a: calcium/calmodulin dependent protein kinase 11a
Tuj1: BIII tubulin
Description of the elements of the invention
UbV.E4B overexpression promotes CST axon regeneration
the inventors firstly discovers that the overexpression of UbV.E4B in cortical spinal cord neurons can promote CST axon regeneration, and further can improve the recovery of a cortical stroke model.

UbV.E4B is a ubiquitin variant, which is a reported inhibitor of Ube4b, capable of inhibiting the activity of Ube4 b.

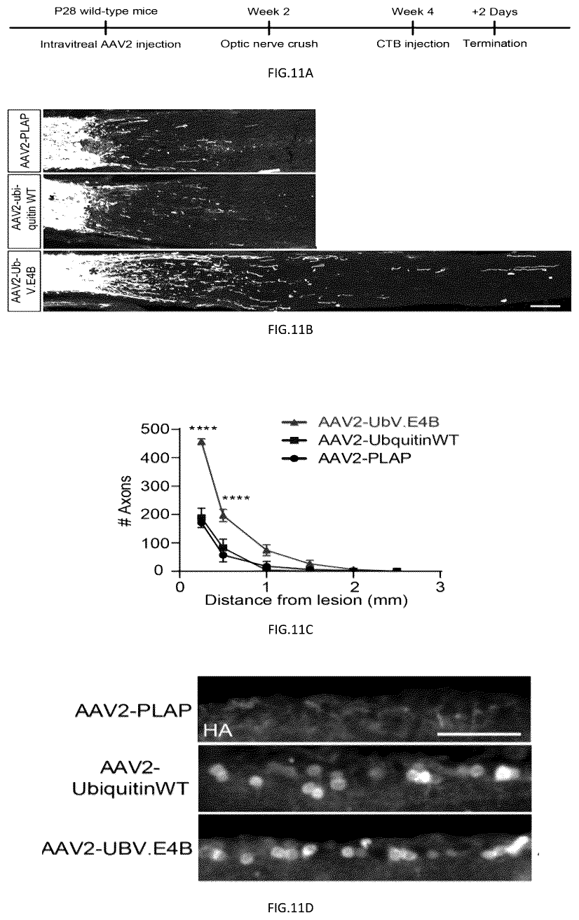

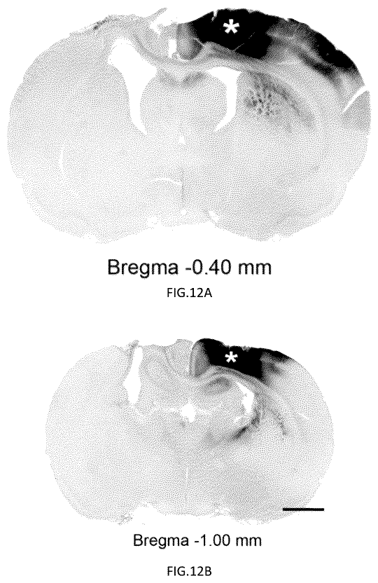

To test the efficacy of UbV.E4B overexpression in CNS axon regeneration, the inventors injected AAV2-PLAP, AAV2-HA-Ubiquitin WT or AAV2-HA-UbV.E4B intravitreal virus into wild-type mice (FIG. 11). The results show that overexpression of UbV.E4B in mature RGC promotes robust optic nerve regeneration after ONC compared to RGC with HA-Ubiquitin WT or PLAP overexpression (FIG. 11B, C, D).

The related sequences are as follows:

| Related gene | Sequence |
|---|---|
| UbV.E4B | ATGTGGATTTTTGTTCAGACCCTGATGCGTAACACCATTACCCTGGAAGT |
| | GGAACCGAGCGATACCATCGAAAATGTTAAAGCAAAAATCCAGGATAAA |
| | GAGGGTATTCCGCCGGATCAGCAGTTTCTGATTTTTACCGGTACCCTGCT |
| | GGAAGACGGCCGTACACTGAGCGATTATAATATTCAGAAAGAAAGTCTG |
| | CTGCGCCTGGTTTGGCGTCCGCGTGGCCCTCTGAATTAA |
| Ubiquitin WT | CTTCAGATCTTCGTGAAGACTCTGACTGGTAAGACCATCACCCTCGAGGT |
| | GGAGCCCAGTGACACCATCGAGAATGTCAAGGCAAAGATCCAAGATAAG |
| | GAAGGCATTCCTCCTGATCAGCAGAGGTTGATCTTTGCCGGAAAACAGCT |
| | GGAAGATGGTCGTACCCTGTCTGACTACAACATCCAGAAAGAGTCCACCT |
| | TGCACCTGGTGCTCCGTCTCAGAGGTGGGTGA |

The above sequences are prior art and no computer readable form sequence listing is provided.

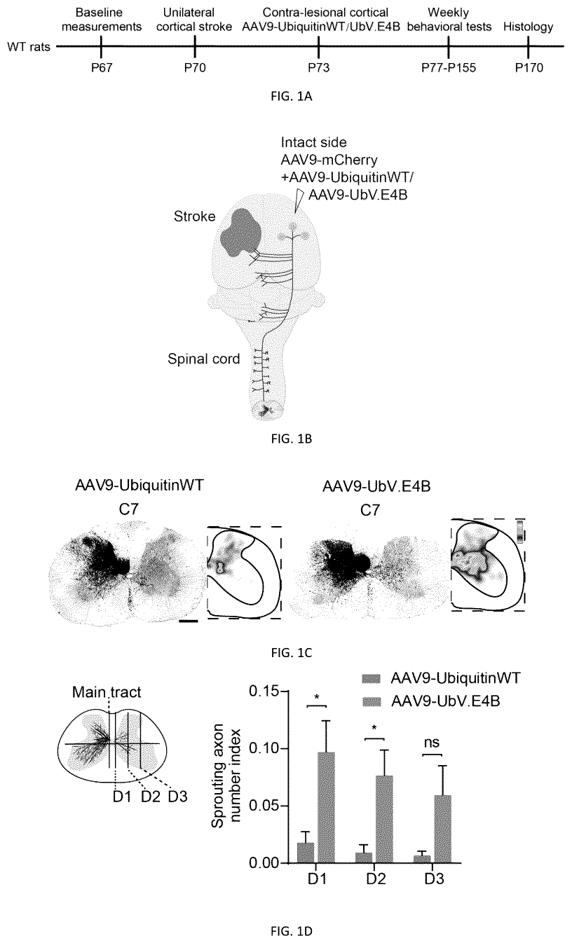

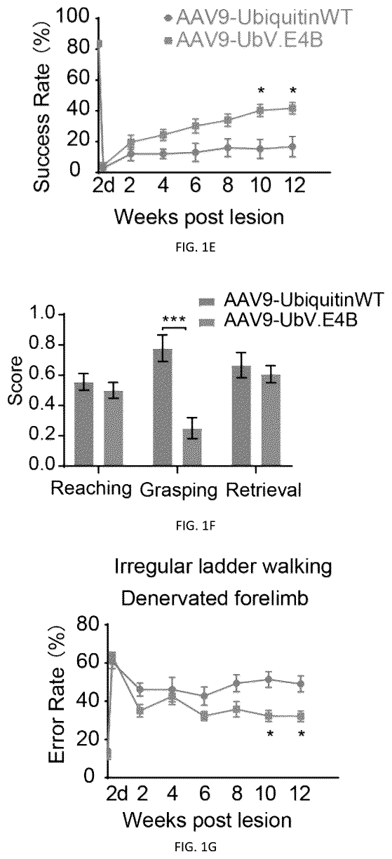

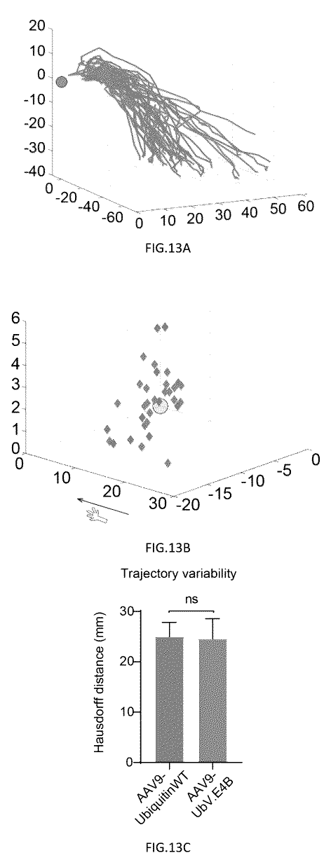

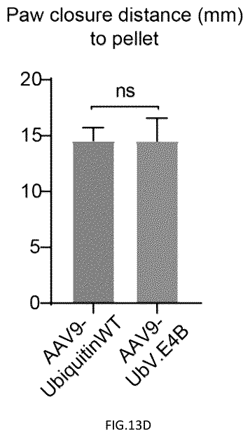

After confirming the efficacy of UbV.E4B overexpression in promoting optic nerve regeneration after ONC, the inventors next sought to assess whether this treatment could also promote CST axon regeneration and associated functional recovery after cortical stroke. For this, adult rats were first trained to master single-grain grips and irregular ladder-walking tasks (FIG. 1A), where CST is critical for the innervation of dexterous movements. the inventors then destroyed one side of CST axons by cortical stroke and injected AAV9-HA-UbV.E4B to the intact side of rat sensory-motor cortex and AAV9-UbiquitinWT as a control (FIG. 1B). The difference in injury-induced regeneration between control and UbV.E4B treated mice is shown by color-coded heatmaps, representing the density of CST regeneration in the contralateral denervated spinal cord (FIG. 1C). the inventors found that, with extensive expression of HA-UbV.E4B in the cortex (FIG. 12A), UbV.E4B treated sensorimotor cortex showed increased regeneration of the cervical-spinal ipsilateral spinal cord, with more axons extending across the midline to the contralateral denervated spinal cord (FIG. 1C, D). In other words, CST axon regeneration to the ipsilateral spinal cord was significantly increased in UbV.E4B treated rats compared to limited regeneration in the control group (FIG. 1C, D). Behaviorally, rats treated with UbV.E4B showed significant recovery of forelimb function in both tasks (FIG. 1E, G), indicating that CST axon regeneration contributes to the improvement of fine motor function of the forelimb after stroke. More specifically, UbV.E4B treatment significantly improved the overall success of the dragee trials (FIG. 1E). The gripping portion of the ball grab test contributes mainly to the observed recovery of forelimb function, rather than the extension or retrieval phase (FIG. 1F), according to a score based on the Eshkol-Wachmann motor notation (EWMN). Furthermore, even though their arrival trajectories were highly variable after stroke (FIG. 13A), there was no significant difference in trajectory variability compared to the control group (FIG. 13C). And the endpoint distribution showed no significant difference between groups (FIG. 13B, D), further verifying that the functional recovery observed in the pellet grasping task was mainly due to the better performance of the rats during the grasping phase.

Based on the above findings, the invention provides the use of UbV.E4B in medicaments for promoting central nerve regeneration.

To confirm that UbV.E4B protein is based on selective inhibition of Ube4b ubiquitination factor, thereby promoting axon regeneration in the central nervous system, rather than achieving a function of promoting nerve regeneration through other pathways the inventors further designed the following experiments.

Experiment for identifying Ube4b as key regulatory factor for inhibiting central nerve axon regeneration

A series of ubiquitin genes including HAUSP, Pirh2, COP1, MDM4, MDM2 and Ube4b were screened. In pilot experiments, the inventors used the optic nerve crush injury (ONC) model to study their role in regulating central nerve axon regeneration, since the optic nerve's anatomy is relatively simple and the disclosure of the optic nerve injury model is also validated by other axon injury models. To this end, the inventors delivered sgRNA and Cre to the retina of Rosa26-Loxp-Stop-Loxp-Cas9 gene knockout mice (LSL-Cas 9 mice) by intravitreal injection of AAV serotype 2 vector (AAV2), which were knocked out separately by CRISPR/Cas9 technology. the inventors demonstrated gene knockout efficiencies in such Retinal Ganglion Cells (RGCs) of >90%.

The sequence of sgRNA is as follows.

| Name of Gene | sgRNA sequence |
|---|---|
| Ube4b | sgRNA 1: AACTGACGCCTTCGCTGCTC |
| | sgRNA 2: ATCTCTGTCGCGGTCCTTCC |
| | sgRNA 3: TTCCCGAATCCACATCCACC |
| | sgRNA 4: CCTGTGTAGGAGTTGCCCAT |
| | sgRNA 5: CTCTTCTGTAGATTCGACGG |
| Pirh2 | sgRNA 1: TGCCTAACCACGAATCTTCG |
| | sgRNA 2: GCTAGATCGTTTCAAAGTCA |
| | sgRNA 3: GTGACAAGCTTTATACCTGC |
| | sgRNA 4: GAATTGTCCAATATGCTTGG |
| | sgRNA 5: TACAGTCTTCACAAGTCTGC |
| Cop1 | sgRNA 1: ATACGGTGCTACTCACTGAG |
| | sgRNA 2: GATGGTCTTACCAAAAGCTG |
| | sgRNA 3: AAAGACTCACCCAAGAAATT |
| | sgRNA 4: CAAAATGTGGCCACAGCTTT |
| | sgRNA 5: CAAGTGTATTCATCAGAGTT |
| HAUSP | sgRNA 1: CAAAAACTTACGCAACTCCA |
| | sgRNA 2: AGAACAAGTGGCTGATTCGC |
| | sgRNA 3: TCTTCAGCACTGCTTGTGCG |
| | sgRNA 4: TGCCTGTACAAAAACTTCAA |
| | sgRNA 5: AGACACCAGTTGGCGCTCCG |
| MDM4 | sgRNA 1: TCCAAGTCAAGACCGACTGA |
| | sgRNA 2: ACATCAGCTTCTATTAACAC |
| | sgRNA 3: ATGTAGGTAATGCACTATCT |
| | sgRNA 4: ACCTTCAGTCGGTCTTGACT |
| | sgRNA 5: GAAGCAGCTCTATGATCAAC |
| MDM2 | sgRNA 1: CGACACTTACACTATGAAAG |
| | sgRNA 2: TTGAAGTTGTTAAAGTCCGT |
| | sgRNA 3: ACCTCTTAGTCATAATATAC |
| | sgRNA 4: TATTGTTCAAATGATCTCCT |
| | sgRNA 5: TTCAGATCACTCCCACCTTC |
| PTEN | sgRNA 1: ACCGCCAAATTTAACTGCAG |
| | sgRNA 2: GCAGCAATTCACTGTAAAGC |
| | sgRNA 3: TGTCATCTTCACTTAGCCAT |
| | sgRNA 4: ACAATATTGATGATGTAGTA |
| | sgRNA 5: AATCCCATAGCAATAATATT |
| Klhl22 | sgRNA 1: CTCATTCCGGTGGTACATAA |
| | sgRNA 2: ACAGTTGTACATCTCGTCAG |
| P53 | sgRNA 1: TGAGGGCTTACCATCACCAT |
| | sgRNA 2: ATAAGCCTGAAAATGTCTCC |
| Control sgRNA | sgRNA: GCGTCGTGACTGGGAAAACCC |

The above sequences are prior art and no computer readable form sequence listing is provided.

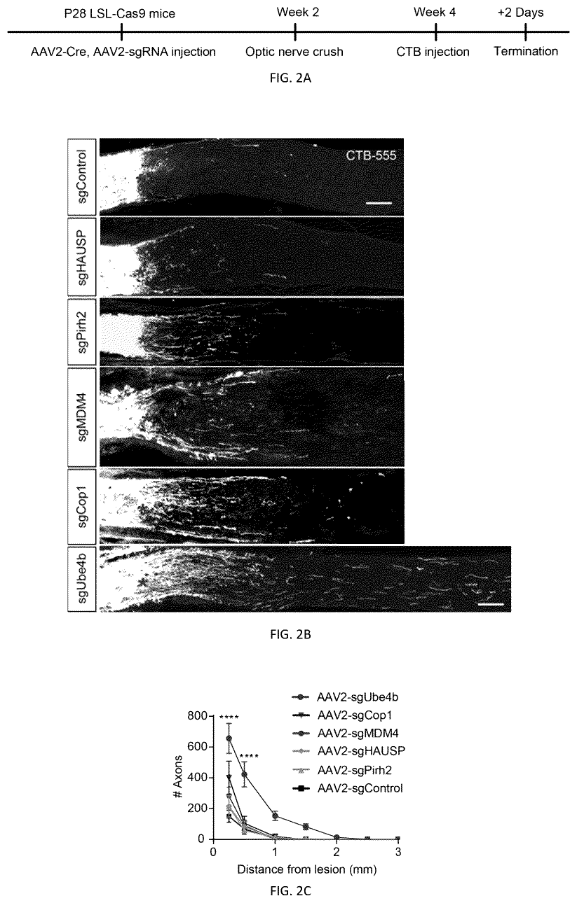

In a control group of LSL-Cas9 mice, the inventors co-injected AAV2-sgControl and AAV2-Cre intravitreally. Two weeks after virus injection, the optic nerve was clipped according to the established protocol 4. After a further 2 weeks, fluorescent conjugated cholera toxin subunit B (CTB) tracer was injected into the vitreous 2 days prior to tissue harvest to monitor axon regeneration (FIG. 2A). Surprisingly, Ube4b gene knockout promoted regeneration of abundant RGC axons after injury, and the regenerated axons extended to about 1.5 mm from the lesion (FIG. 2B). This is in complete agreement with UbV.E4B overexpression by promoting CST axon regeneration.

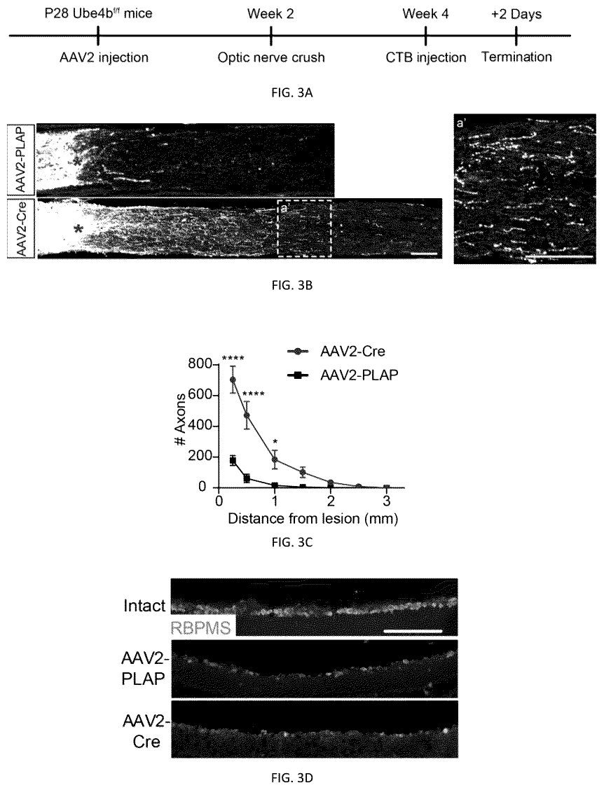

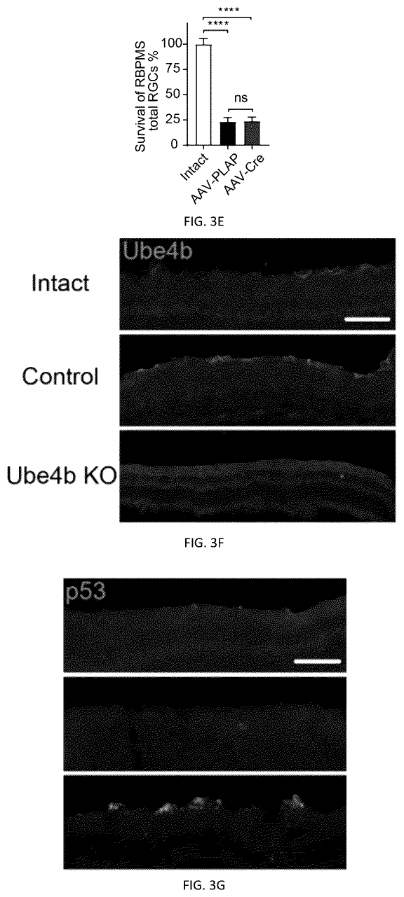

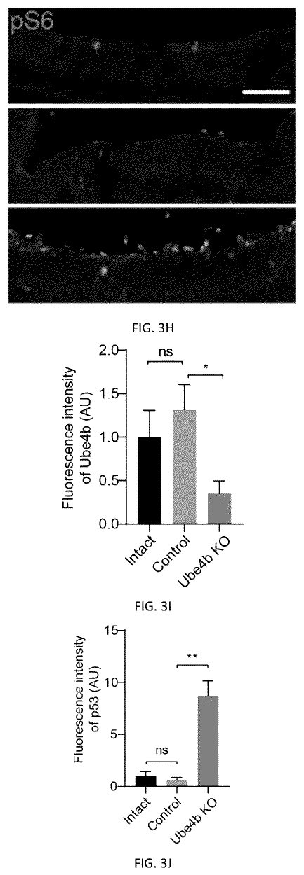

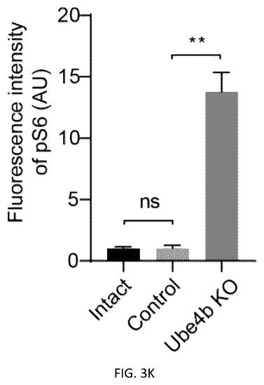

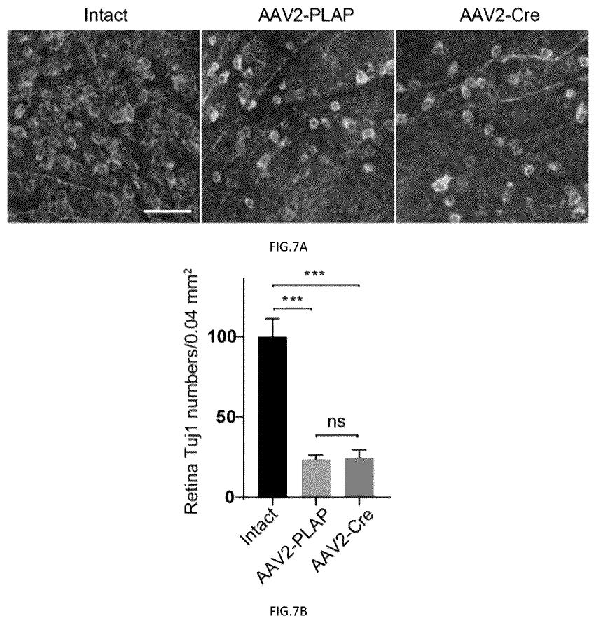

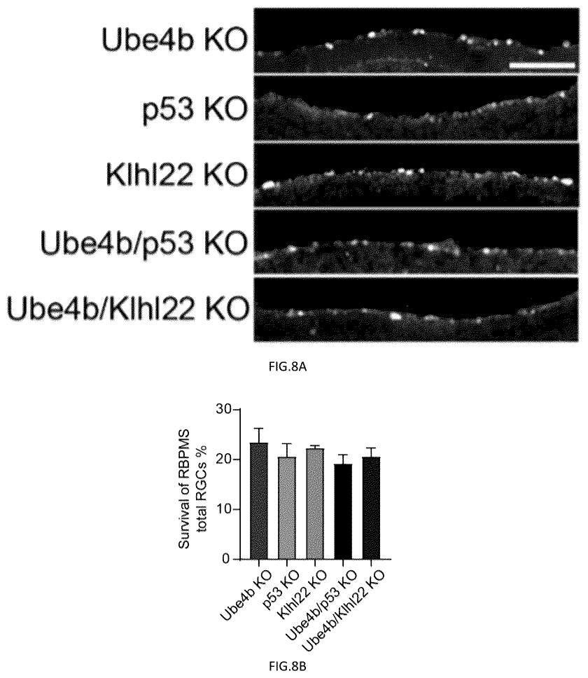

In view of the knockout efficiency and potential off-target issues of the CRISPR/Cas9 system, the inventors constructed a transgenic mouse strain with a homologous conditional Ube4b mutant (Ube4bf/f) to further validate the effect of Ube4b knockout on axon regeneration (FIG. 3A). By intravitreal injection of AAV2-Cre or AAV2-PLAP controls to Ube4bf/f mice, deletion of Ube4b in RGCs significantly increased optic nerve axon regeneration compared to controls (FIG. 3A-C), consistent with results obtained with CRISPR/Cas9 knockout technology. Since gene knockout may also affect RGC survival, we assessed RGC survival 2 weeks after AAV2-PLAP and Ube4b knockout mouse ONC injury by whole retinal staining of the RGC-labeled antibodies RBPMS and Tuj1. the inventors found that survival of RGC was not affected by the Ube4b gene knockout (FIG. 3D, e and FIG. 7A, B), suggesting that the Ube4b gene knockout promotes optic nerve regeneration by increasing the regenerative potential of RGC surviving in ONC, rather than by protecting RGC from death of ONC.

Based on the same principle, the invention also provides the use of a nucleic acid sequence for encoding UbV.E4B protein in preparing medicaments for promoting central nerve regeneration, which is characterized in that the Ube4b ubiquitination factor is selectively inhibited by obtaining the protein through expressing UbV.E4B gene, so that central nervous system axon regeneration is promoted.

### Research on Mechanism for Promoting Nerve Regeneration by Knocking Out Ube4b

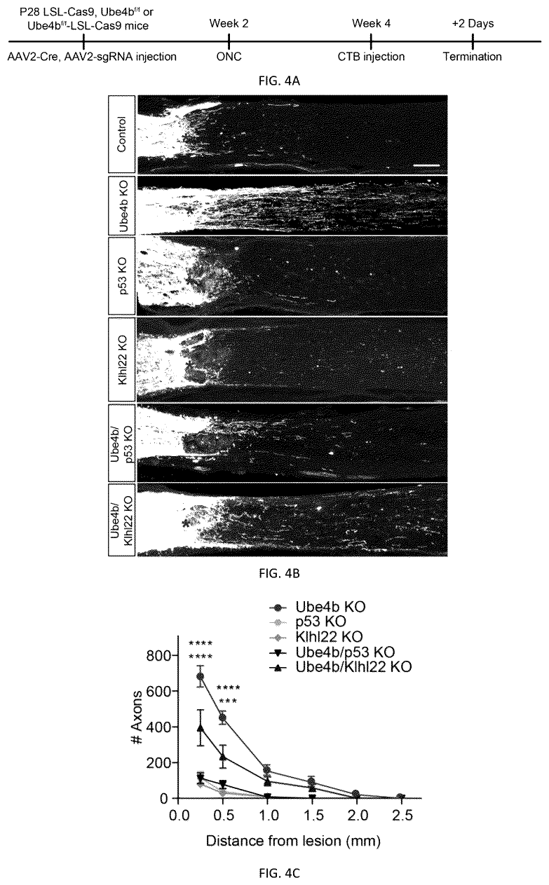

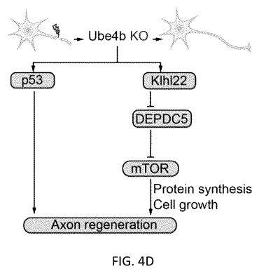

the inventors confirmed based on the following experiments that deletion of Ube4b promotes axon regeneration at least by altering the activity of p53 and mTOR. Two weeks after ONC the inventors immunostained retinal sections with Ube4b, p53 and pS6 (an indicator of mTOR activity). As expected, the inventors observed a clear downregulation of Ube4b (FIG. 3F, I) and upregulation of p53 (FIG. 3G, J) in the knockout RGCs of Ube4 b. Surprisingly, the inventors found that there was a significant up-regulation of pS6 levels in ONC following Ube4b knockout (FIG. 3H, K), indicating that Ube4b could simultaneously regulate both p53 and mTOR pathways after ONC injury. Proteomic results show that the deletion of Ube4b results in significant upregulation of Klhl22, and the inventors speculate that the deletion of Ube4b may increase RGC regeneration potential through the Khl22/mTOR pathway. To directly investigate the contribution of p53 or Khl 22-dependent pathways to Ube4b deletion on the effect of axon regeneration, the inventors crossed Ube4bf/f mice and LSL-Cas9 mice, generating pure and mutant carrying two flox genes (Ube4b and Cas9). Mice with double mutants received intravitreal viral injections of AAV2-Cre and/or AAV2-sgRNA to induce deletion of one or both genes in RGCs (FIG. 4A). The results showed that p53 or Khl22 knockout did not significantly alter the regenerating phenotype of injured optic nerve axons compared to controls (FIG. 4B, C). However, double knockout of Ube4b and p53/Klhl22 showed a significant loss of axon regeneration observed in mice deleted for Ube4b (FIG. 4B, C), indicating that deletion of p53 and Klhl22 partially abolished the axon regeneration phenotype elicited by Ube4b deletion. Furthermore, there was no statistical difference in neuronal survival between the Ube4b, p53, Khl22, and double gene knockouts (FIG. 8A, B), indicating that apoptosis of non-RGCs impairs axon regeneration. Finally, the inventors demonstrated that the deletion of Ube4b in RGCs promotes robust axon regeneration after optic nerve injury in a dual pathway-dependent manner through p53 and Khl22/mTOR (FIG. 4D).

Based on the experiments, the Ube4b inhibitor promotes central nerve axon regeneration by simultaneously regulating dual pathways of mTOR and p53, so that the effect of Ube4b in promoting nerve regeneration is quite obvious.

In a preferred embodiment of the invention, the Ube4b ubiquitination factor is selectively inhibited through expression, so that the dual pathways of mTOR and p53 are simultaneously regulated, and the axon regeneration of the central nervous system is promoted.

In a preferred embodiment of the invention, the central nerve axon regeneration is optic nerve regeneration.

Based on the clear and high-efficiency physiological activity of the Ube4b ubiquitination factor, the Ube4 ubiquitination factor can be used as a target for screening the molecular level of the nerve regeneration medicaments, and has unprecedented advantages compared with screening medicaments with nerve regeneration promoting effects by using animal models. Therefore, the invention provides an application of the Ube4b protein as a target substance in a medicament screening substance for promoting central nervous system axon regeneration.

In a preferred embodiment of the present invention, the agent for promoting central nerve regeneration is an inhibitor of Ube4b protein. In a preferred embodiment of the present invention, the inhibitor of Ube4b protein can be protein, antibody, nucleic acid medicament, small molecule medicament.

It is understood that substances capable of allosterizing Ube4b, substances capable of competitively occupying ubiquitin sites of Ube4b protein (for example, proteins obtained by expressing UbV. E4B gene), and substances capable of inhibiting the expression and synthesis of Ube4b in vivo all have potential as medicaments for promoting central nervous system axon regeneration, and that medicaments having the function of promoting central nervous system axon regeneration can be obtained by screening these substances.

the inventors have also designed the following experiments in order to seek synergy with other possible pathways.

### Ube4b and PTEN Double-Gene Knockout Further Promotes Optic Nerve Regeneration

the inventors explored the synergistic effect of Ube4b and PTEN double gene knockouts in axon regeneration. In Rosa26-LSL-Cas9 mice, the inventors used AAV2-Cre to knock out PTEN by co-injection with AAV2-sgPTEN in vitreous (PTEN knockout group). In the Ube4bf/f/LSL-Cas9 mouse, the inventors combined knockout of PTEN and Ube4b (Ube 4b/Pten knockout group) by intravitreal injection of AAV2-Cre and AAV2-U6-sgPTEN (FIG. 4A). the inventors found that double knockout of Ube4b and PTEN induced a stronger optic nerve regeneration phenotype compared to PTEN alone (FIG. 4B, C). Specifically, the combined knockout of Ube4b and PTEN induced more significant axon regeneration at 500, 750, 1000, 1500, 2000, 2500, 3000 and 3500 μm from the injury site, compared to the knockout of Ube4b or PTEN alone, even with many axons reaching visual cross within 2 weeks after injury, which was rare after treatment alone (FIGS. 2C and 4D). Notably, PTEN deletion increased survival of RBPS+RGCs in Ube4b/PTEN-co-deleted mice to about 50% (FIG. 4E, F). These results indicate that co-deletion of Ube4b and PTEN results in greater regenerative capacity than deletion of PTEN alone or Ube4b, probably because, in addition to the mTOR pathway, Ube4b knockout recruits the p53 pathway to further improve the axonal regenerative capacity of RGCs after ONC injury.

The experimental result shows that the method of inhibiting transcription or expression products of PTEN gene while adopting the strategy of inhibiting Ube4b can obtain more excellent effect on promoting optic nerve regeneration.

### Ube4b and MDM2 Double-Gene Knockout Effect on Promoting Optic Nerve Regeneration

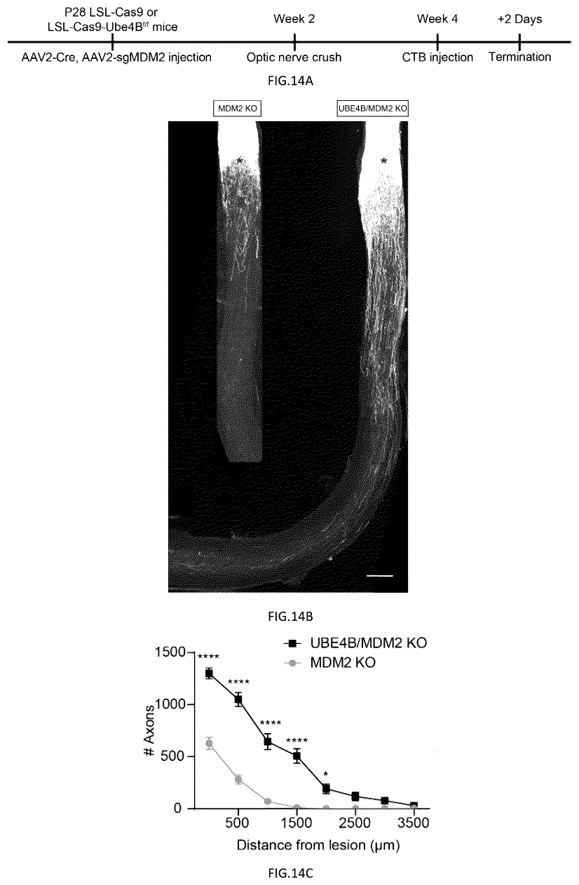

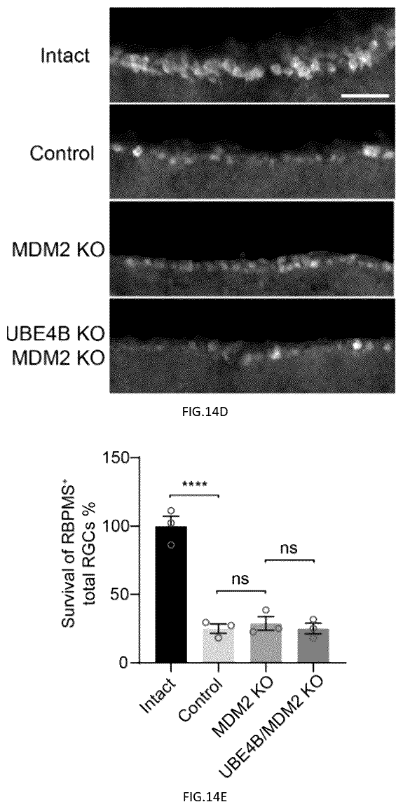

Similar to the Ube4b and PTEN double gene knockouts described above, MDM2 was knocked out by intravitreal injection of AAV2-Cre and AAV2-U6-sgMDM2, and the loss of MDM2 was shown to lead to axon regeneration following optic nerve injury (see FIG. 14A-E). Meanwhile, the inventors knockout MDM2 and Ube4B in UBE4B f/f-LSL-Cas9 mice by intravitreal injection of AAV2-Cre and AAV2-U6-sgMDM2. Interestingly, the knockouts of Ube4B and MDM2 achieved more robust regeneration than either Ube4B or MDM2 alone, and did not affect survival of RGC (see FIG. 14A-E).

This suggests that inhibitors of transcription or expression of the MDM2 gene could be synergistic with the neuroregenerative therapy of the invention, and such inhibitors also include small molecule pathway inhibitors, such as the well-known MDM2-p53 inhibitor APG-115.

the inventors further performs the following experiments to confirm the excellent technical effects of the technical scheme of the invention.

Ube4b Gene Knockout Promotes CST Axon Regeneration in Spinal Cord after Unilateral Cortical Stroke

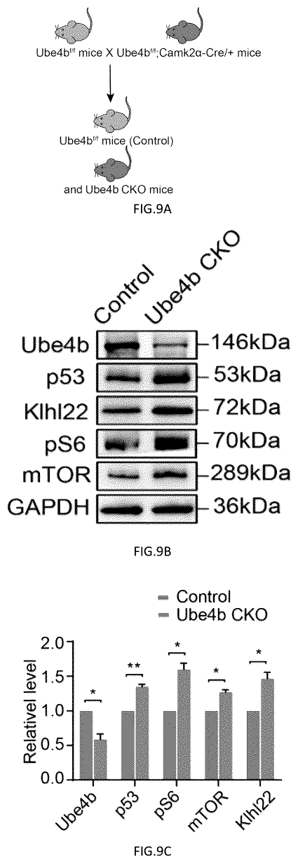

After verifying that Ube4b deletion in RGCs can promote axon regeneration following injury, the inventors attempted to evaluate whether this strategy is also effective for cortical neurons. Prior to carrying out cortical axon regeneration studies, the inventors first attempted to assess whether the molecular pathways observed in RGCs are also involved in Ube4b knockout cortical neurons. To this end, the inventors crossed the Ube4bf/f mice and Camk2a-Cre mice, generating mutant mice in which Ube4b could be specifically knocked out in Camk2a-neurons (FIG. 9A-C). Immunoblot studies on these cortical neurons demonstrated that deletion of Ube4b also upregulated the levels of p53, Khl22, pS6 and mTOR (FIG. 9B, C), consistent with the results for Ube4b knockout RGCs. This result demonstrates that Ube4b knockout can also modulate the p53 and mTOR pathways of cortical neurons.

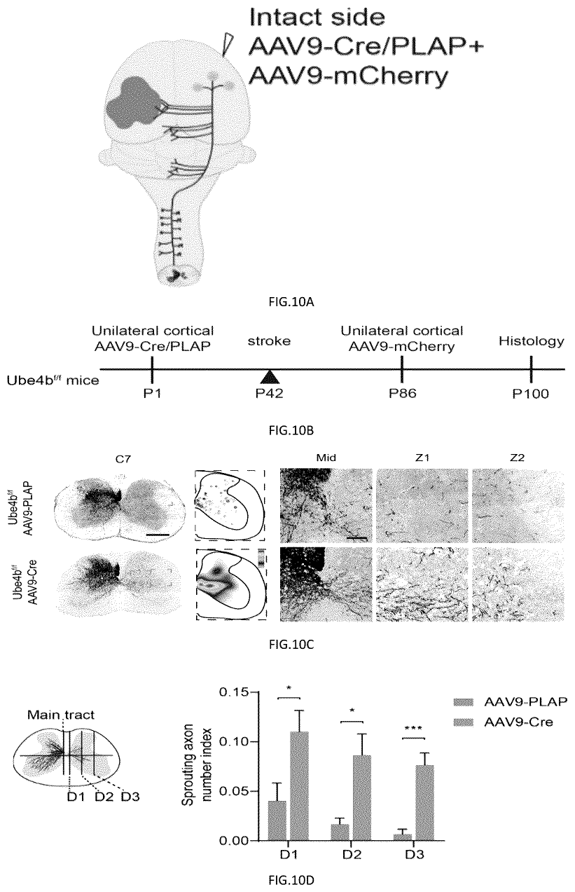

With these results, the inventors next attempted to assess whether deletion of Ube4b could promote regeneration of corticospinal tract (CST) axons in a cortical stroke model. To this end, the inventors first injected either AAV9-Cre (Ube4b knockout) or AAV9-PLAP (control) into the right sensorimotor cortex of newborn Ube4bf/f mice (FIG. 10A, B). After 6 weeks of virus injection, the inventors performed unilateral cortical stroke on left sensorimotor cortex, destroying CST axons with minimally invasive, reproducible photochemical cortical lesions (FIG. 10A, B). AAV9-mCherry was injected into the intact sensorimotor cortex of mice 6 weeks after unilateral cortical stroke, where mCherry was used to label axons from the intact sensorimotor cortex. The difference in CST axon regeneration between control and Ube4b-deleted mice can be seen by color-coded heat maps of axonal sprouting density. The Ube4b-deleted sensorimotor cortex showed an increase in Mid, Z1 and Z2 in the cervical spinal cord towards ipsilateral spinal cord regeneration compared to the limited regeneration in the control group (FIG. 10C). In Ube4b knockout mice, more axons extended through the midline to D1, D2 and D3 (FIG. 10D). Finally, the inventors showed that the deletion of Ube4b significantly enhanced adult CST axon regeneration after cortical stroke.

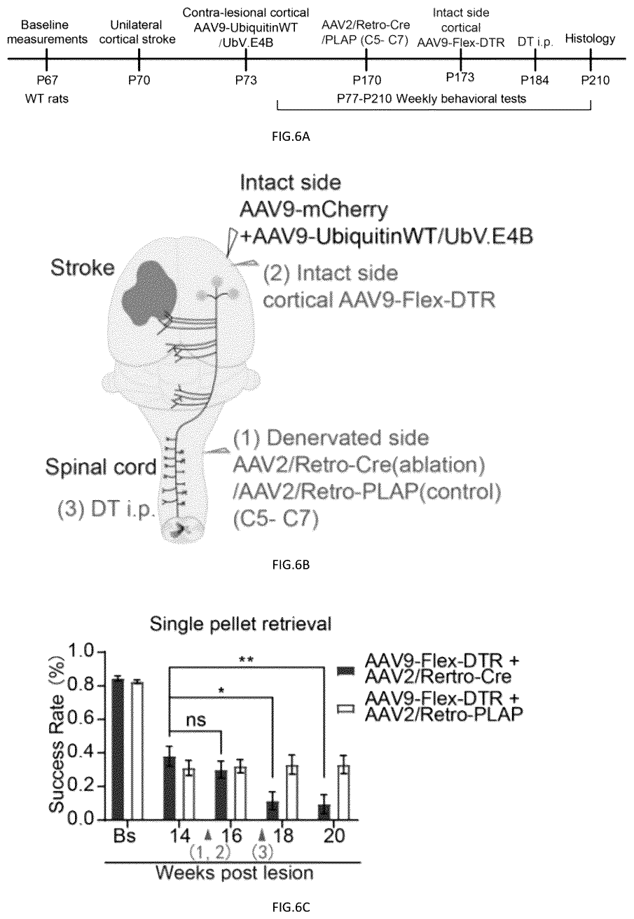

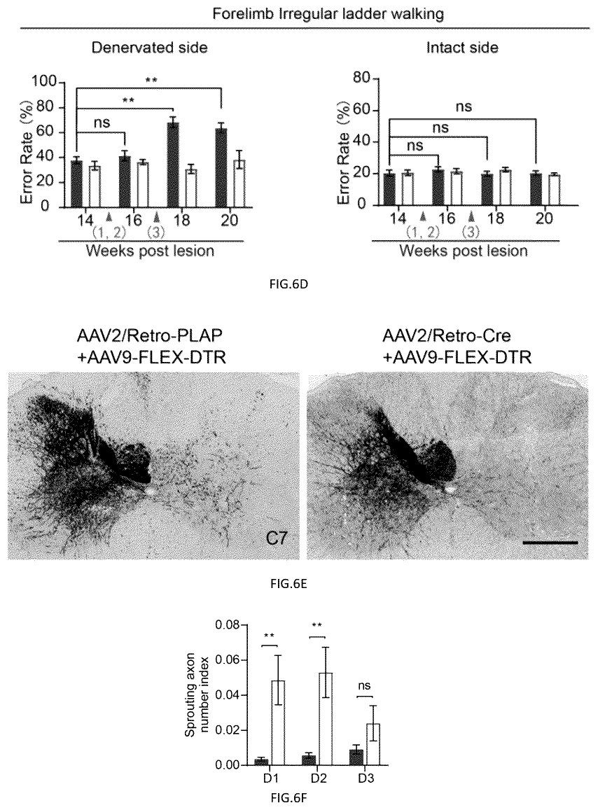

the inventors found that ablation of regenerated CST axons abolished the recovered proficient motor performance. After injecting AAV2/retro-Cre unilaterally to the denervated side of the cervical spinal cord, the inventors injected AAV9-Flex-DTR (ablative) or AAV9-Flex-PLAP (control) into the intact lateral cortex of a group of UbV.E4B expressing rats as described above, selectively killed regenerated axons in the cervical spinal cord (C5-C7) by intraperitoneal injection of diphtheria toxin DT (FIG. 6A, B). the inventors demonstrated that these intraspinal and cortical injections did not alter the behavioral performance of rats (FIG. 6C, D). However, the improved performance in the dragee-grasping task and irregular stepped walking of denervated forelimbs by expression of UbV.E4B was significantly reduced 2 weeks after DT administration (FIG. 6C, D). Consistently, ablation of CST axons was observed on the denervation side of the cervical section (FIG. 6E, F). Thus, the results of the inventors' studies indicate that regenerated axons in the spinal cord are necessary to restore skilled motor performance after unilateral photothrombosis stroke. The experiment further knockout the gene of Ube4b ubiquitination factor is consistent with the effect of inhibiting the function of the gene.

The invention also provides a pharmaceutical composition for promoting the regeneration of central nerves, which comprises a Ube4b ubiquitination factor inhibitor and a pharmaceutically acceptable carrier.

In a preferred embodiment of the invention, the Ube4b ubiquitination factor inhibitor is UbV.E4B protein or a vector comprising a nucleic acid sequence encoding UbV.E4B protein. It may be formulated for administration to the brain, spinal cord, or optic nerve.

The pharmaceutical compositions of the present invention may be formulated for administration by an intracerebroventricular, intranasal, intracranial, intraventricular, intracerebellar, or intrathecal route of administration.

In conclusion, the inventors discovered that Ube4b is a key regulator by using in vivo CRIPR screening and gene manipulation strategies through the clue of UbV.E4B protein overexpression to achieve nerve regeneration, and Ube4b simultaneously regulates two main pathways, namely p53 and mTOR, for controlling optic nerve and CST axon regeneration. The AAV-based UbV.E4B protein overexpression method has wide clinical application prospect, and can realize a transformation strategy for recovering CST dependent function after cortical stroke.

Surprisingly, Ube4b plays a major role in regulating central nervous system axon regeneration in the ubiquitin pathway. The Ube4b deletion showed a stronger effect on axon regeneration and was achieved in multiple ways.

Previously, strategies such as activation of mTOR and or overexpression of p53 have been proposed to promote central nervous system axon regeneration. mTOR signaling pathway is thought to play an important role in synaptogenesis, differentiation, and in particular, axon regeneration and neuronal survival after central nervous system injury. p53 is a well-known tumor suppressor and is a multifunctional sensor of several cellular signals and pathways, essential for angiogenesis, cellular metabolism, DNA damage, cell cycle regulation, apoptosis and nerve regeneration. However, key molecules that regulate both of these important mechanisms have not been identified. In this study, the inventors showed that Ube4b is involved in central nervous system axon regeneration, whereas Ube4b has been shown to promote polyubiquitination and degradation of p53 and inhibit p53-dependent transactivation and apoptosis. Further studies have shown that, in addition to p53, Ube4b recruits the mTOR pathway through its substrate Khl22 to promote axon regeneration, and thus explores a new mechanism for controlling central nervous axon regeneration.

In the rat cortical stroke model, overexpression of UbV.E4B in cortical neurons was shown to restore CST-dependent behavioral recovery by promoting CST axonal sprouting. Although many intrinsic mechanisms controlling axon regeneration in the central nervous system have been explored, few people evaluate them in a transformable environment. Here, in a transformable environment, the inventors showed that overexpression of UbV.E4B was sufficient to trigger strong regeneration and functional recovery of CST axons, suggesting the potential to provide the UbV.E4B protein as a treatment for cortical infarction. However, regrown CST axons may need to be fine-tuned for better function. Since motor and electrical stimulation can promote plasticity of CST axons towards the functional spinal network, combining electrical stimulation with UbV.E4B overexpression therapy may further improve functional recovery after cortical stroke.

### Expression and Purification of UbV.E4B Recombinant Protein and Treatment of Central Nervous System Injury

Despite the advantages of AAV vector-based gene therapy, AAV vector-based gene therapy has some challenges, such as limited capacity of packaging gene of AAV vector and difficulty of AAV passing through blood-brain barrier, so that AAV vector-based gene therapy has limited treatment of diseases such as central nervous system, and delivery efficiency in some organs is still to be improved, and there are many problems in clinical application due to high treatment price. In consideration of potential risks of AAV gene therapy in clinical application, the expression and purification of UbV.E4B recombinant protein is attempted and the effect on the treatment of central nervous system injury is directly tested.

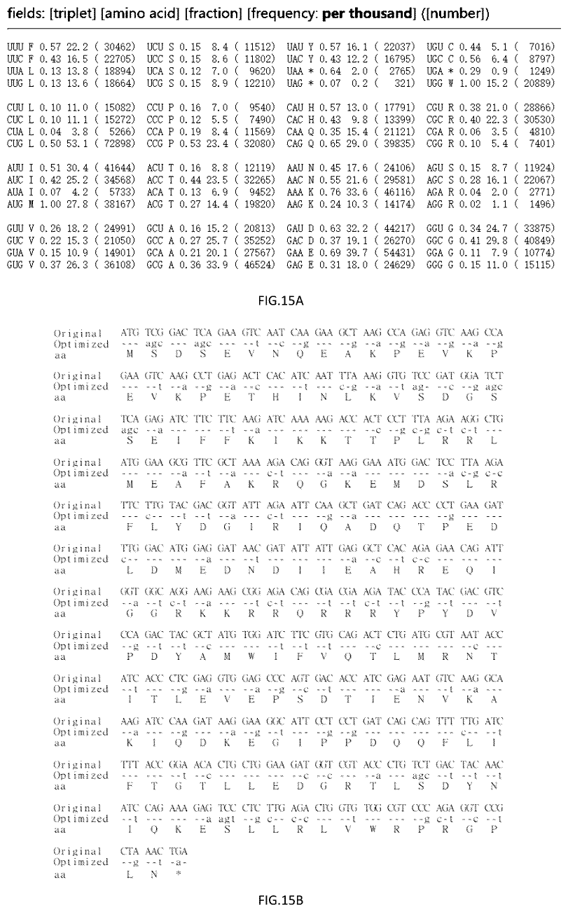

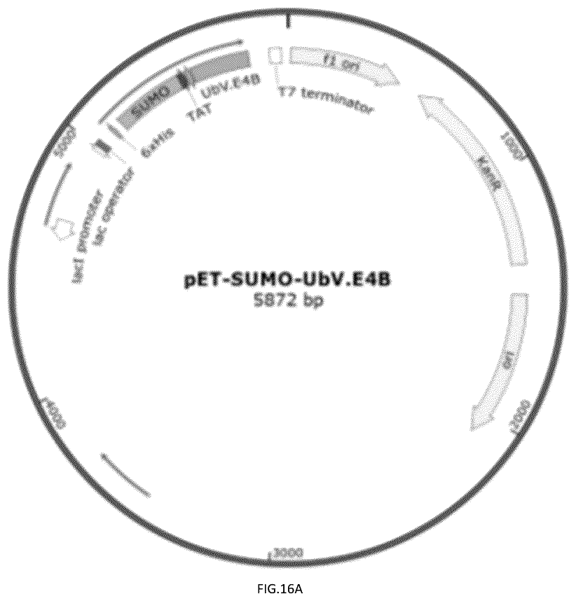

Firstly, the inventors utilized an Escherichia coli recombinant expression system to express rUbV.E4B, and the control is recombinant protein UbiquitinWT (rUbiquitinWT). In order to increase the expression level of the UbV.E4B gene in E. coli BL21 (DE3)/pLysS, the inventors first performed codon optimization of the UbV.E4B gene with a tag. According to the codon preference use database of Escherichia coli (see FIG. 15A), all codons with low codon usage frequency in the UbV.E4B gene were changed to codons with high usage frequency, but the coding sequence of the UbV.E4B gene was not changed. As shown in FIG. 15B, a total of 122 codons in amino acids of SUMO-TAT-HA-UbV.E4B gene were optimized (Database: http://www.kazusa.or.jp/codon/cgi-bin/showcode.cgi?species=413997). And comparing the sequences of the UbV.E4B gene after codon optimization and the original UbV.E4B gene, wherein the base parts marked by the Optimized lines are Optimized codon bases.

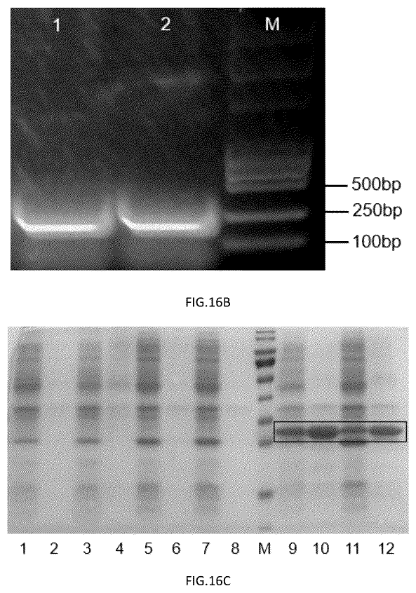

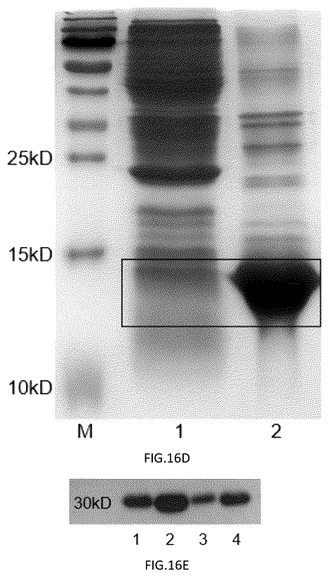

Next, the inventors constructed pET-SUMO-UbV.E4B expression vectors. The inventors synthesized the vector pET-His-SUMO-TAT-HA-UbV.E4B (hereinafter referred to as pET-SUMO-UbV.E4B) (FIG. 16A-16D). Two single colonies were picked from the transformed Escherichia coli DH5a and respectively cultured overnight, bacterial liquid PCR identification was carried out, obvious bands were arranged at the positions of 100-250 bp (FIG. 16A-16D), and the success construction of the vector pET-SUMO-UbV.E4B was proved by combining the sequencing result. Then, the inventors expressed and identified rUbV.E4B. The inventors transformed a correctly constructed pET-SUMO-UbV.E4B vector into an expression host strain BL21 (DE3)/pLysS to obtain an engineering strain E. coli BL21 (DE3)/SUMO-UbV.E4B, and identified the protein expression of the engineering strain. The positive colony transformed by the pET-28a (+) empty vector was used as a negative control, and the SDS-PAGE identification result shows that the protein expression condition of the negative control was basically unchanged before and after IPTG induction. After IPTG induction, compared with blank control, protein expression was detected at 30 kD in the supernatant and inclusion bodies of the engineering strain E. coli BL21 (DE3)/SUMO-UbV.E4B after disruption (FIG. 16A-16D). In addition to expression vectors carrying SUMO, expression vectors without SUMO were constructed in this study. Compared with the expression of pET-UbV.E4B transformed positive recombinant bacteria (E. coli BL21 (DE3)/UbV.E4B) without SUMO tags (FIG. 16A-16D), the protein expression amount collected from the supernatant after disruption E. coli BL21 (DE3)/SUMO-UbV.E4B bacteria was obviously improved, namely the soluble expression efficiency of the recombinant protein containing SUMO was obviously higher than that of an expression vector without SUMO (FIG. 16A-16D), and the recombinant protein fused with SUMO was proved to promote the soluble expression of rUbV.E4B. To further confirm that the protein bands in this study are all rT AT-HA-UbV.E4B, Western blotting analysis of anti-HA monoclonal antibody was performed, indicating that both engineered strains can express the rUbV.E4B protein (FIG. 16A-16D), which is also consistent with the results of the previous study (FIG. 16C, lanes 9, 10, 11 and 12).

The inventors inoculated engineering bacteria E. coli BL21 (DE3)/SUMO-UbV.E4B into 5 ml LB liquid culture medium (Kan+5 μL/ml) for overnight culture, cultures were inoculated into 50 ml LB liquid culture medium containing kanamycin in an inoculation amount of 2%, shake culture was carried out at 37° C. firstly until OD600 is 0.6-0.8, inducer IPTG with the final concentration of 1 mM was added, shake culture was carried out at 30° C. and 230 rpm for 5 hours, bacteria liquid was centrifuged (4° C., 8000 rpm and centrifuged for 15 min), bacteria precipitates were collected, the bacteria bodies were resuspended in 15 ml PBS buffer pre-cooled by ice, ultrasonic disruption and centrifugation were carried out, total bacteria protein in supernatant was collected, and the expression condition of the protein was detected by SDS-PAGE.

Eluents containing imidazole at different concentrations will have some effect on the purity and quality of the recombinant protein collected. When the fusion protein passes through a nickel column for the first time, His-Sumo-UbV.E4B is combined with the column depending on the specific affinity between the His tag and nickel ions, Miscellaneous proteins is washed by buffer solution or low-concentration imidazole, and then the target protein is eluted by imidazole with different gradients, so that the crude and pure target protein can be obtained. High-purity UbV.E4B is further obtained by the following four-step method.

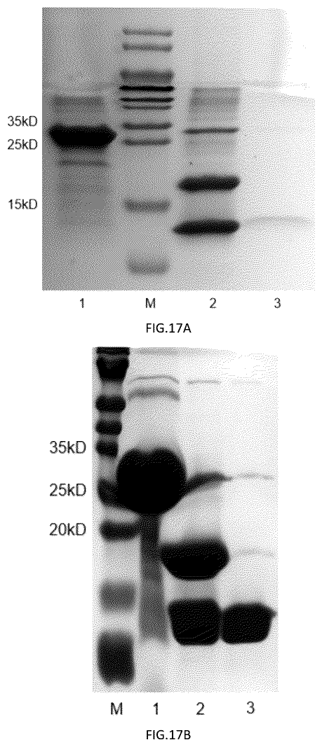

Firstly, an engineering strain E. coli BL21 (DE3)/SUMO-UbV.E4B of Escherichia coli is induced by 1 mM IPTG, crushed bacteria are centrifuged to generate supernatant containing multiple proteins, the supernatant is primarily purified by Ni-NTA affinity chromatography, and the eluate is collected after being eluted by eluate containing 400 nM imidazole. Then, considering that the affinity tag may affect the biological activity of the protein, the inventors cleaved SUMO and His tag from the recombinant protein after the primary affinity purification with SUMO protease, and SDS-PAGE showed that the crude pure sample was cleaved into two bands (FIG. 17A), indicating that SUMO protein cleavage was successful. And secondly, passing the crude pure sample subjected to the SUMO enzyme digestion through a nickel column again to simultaneously remove the His tag and the SUMO protein, and collecting flow-through liquid to obtain the target protein. The recombinant protein is usually eluted by other hetero-proteins through nickel affinity chromatography, so high-purity recombinant protein is obtained by a multi-step purification method, while molecular sieve or molecular exclusion chromatography is one of the most effective methods for separating protein mixtures according to molecular weight, when a sample flows through a column packing, the recombinant protein flows out of the column at a higher speed due to the exclusion of the molecular weight by the packing, and other hetero-proteins, small molecules such as salt ions and the like take a longer time to flow through the column packing before flowing out of the column, so that the inventors utilized Sephadex-G75 gel filtration chromatography for further purification, so that the purity of rUbV.E4B can meet further experimental requirements (FIG. 17A). The band after the secondary separation and purification is single, which shows that the high-purity recombinant protein rUbV.E4B can be obtained by four steps of primary affinity chromatography, SUMO enzyme digestion, secondary affinity chromatography and filtration chromatography. The purity of the obtained recombinant protein rUbV.E4B is up to 90% (FIG. 17A) by analysis of ImageJ software, and the recombinant protein rUbV.E4B can be used for subsequent experiments. Using the same four-step method, the inventors also obtained a high-purity control recombinant protein UbiquitinWT (FIG. 17B). The obtained recombinant protein rUbV.E4B was used for the following study on the influence on nerve regeneration. In order to allow the recombinant protein to pass through the cell membrane into the interior of the cell, the recombinant protein purified by the inventors has a TAT membrane-penetrating peptide tag.

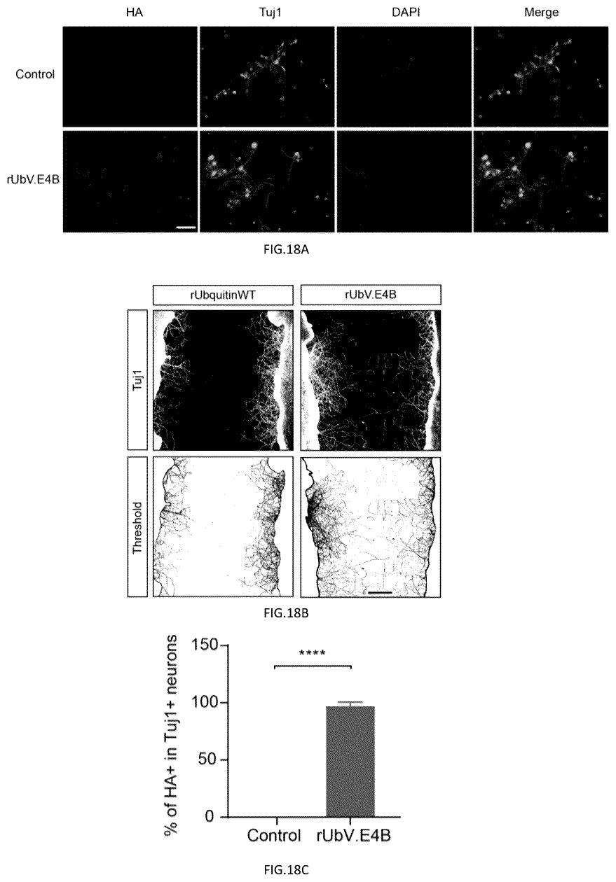

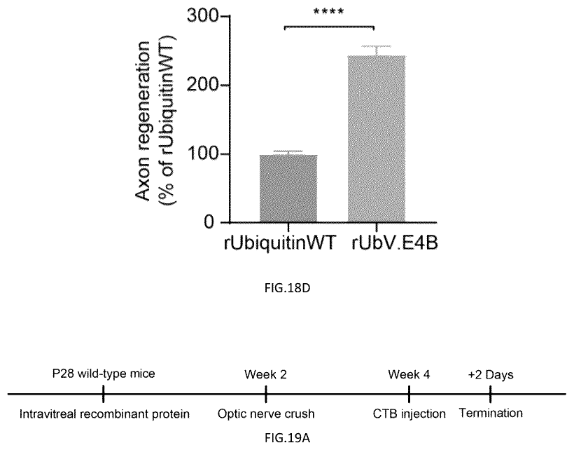

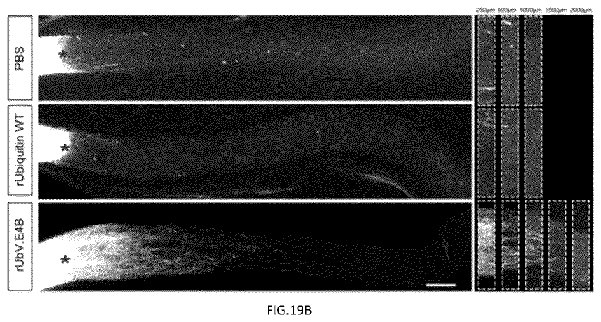

The inventors first verified the efficiency of rUbV.E4B entering cells. rTAT-HA-UbV.E4B was added to neurons cultured in vitro, and PBS was used as a blank control group. One day after treatment, the cells were fixed with PFA and immunostained with HA antibody and neuron-specific antibody Tuj1. As expected, HA and Tuj1 were highly co-labeled (FIG. 18A and C), indicating that the recombinant protein was able to enter the cells. Next, the inventors studied the effect of rUBV.E4B protein on axon regeneration in vitro. The inventors used a scratch test to extract neonatal mouse cortical neurons and inoculate them into a culture dish, after 10 days of in vitro culture, the tip of a pipette was used to scratch the bottom of the culture dish to mechanically damage the axons145, and then 10 ng/uL of rUBV.E4B protein was added to the culture dish, and rUbiquitinWT protein was added to the control group cells. One day after recombinant protein treatment, axon regeneration after scratch injury in the rUBV.E4B-treated group was significantly increased compared with the control group (FIG. 18B and D), suggesting a positive role of rUBV.E4B protein in axon regeneration.

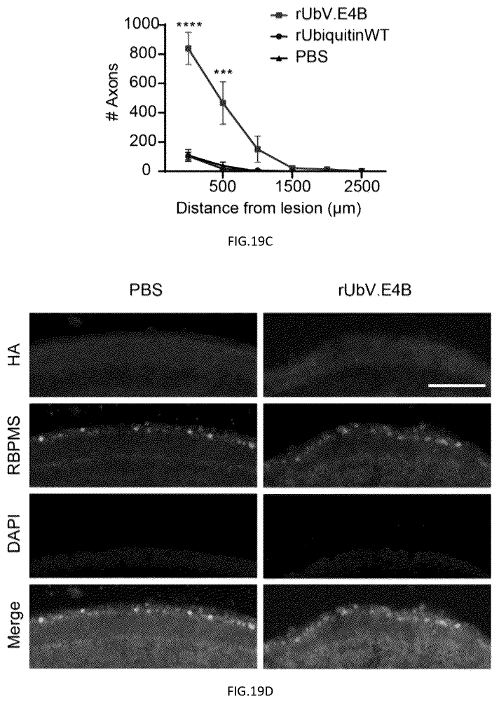

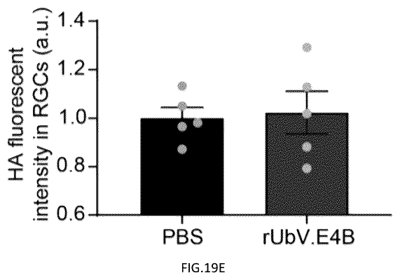

In addition, to explore the effect on axon regeneration after nerve injury, the inventors performed ocular vitreous injection of purified rUbV.E4B protein into 4-week-old wild-type mice, and the control group was injection of rUbiquitinWT. Two weeks after the protein injection, the optic nerve was operated with a clamp wound, two weeks after the injury, CTB was injected and axon regeneration of the optic nerve was observed (FIG. 19A). Morphological results showed that optic nerve regeneration was significantly increased in the rUbV.E4B compared with the control group. Three sets of retinal sections were immunostained for HA-tagging with statistically no difference in fluorescence intensity (FIG. 19D and E), indicating that recombinant protein may have degraded 4 weeks after injection. The above results suggest that rUbV.E4B protein can promote regeneration after central nervous system injury, but solving the problem of protein degradation may help regenerate more axons and longer distances.

The influence of the recombinant protein UbV.E4B on nerve regeneration is researched by means of protein expression and purification and in vitro and in vivo experiments, and the following conclusion is obtained: rUbV.E4B is capable of penetrating cell membranes into cells; rUbV.E4B can promote axon regeneration of in vitro cultured cells; rUbV.E4B is capable of promoting optic nerve regeneration in vivo. The effect of exogenous recombinant protein for promoting regeneration is not obvious due to in vivo metabolism and the like, however, the inventors discovered that rUbV.E4B protein with the cell-penetrating peptide designed by the invention has unexpectedly excellent effect and duration for promoting nerve regeneration.

### Supplementary Description of Specific Experimental Methods

The specific experimental procedure is recorded below.

### Experimental Model

### Mouse Strain

Primary mouse strains include fl-Ube4b-fl and Rosa26-LSL-Cas9, provided by the Wangzhiping laboratory of Zhejiang University School of Medicine. Rosa26-LSL-Cas9 hybridized with fl-Ube4b-fl, yielding fl-Ube4b-fl and fl-STOP-fl-Cas9-GFP homozygotes (harvested from F2). By transferring CRE recombinant protein and AAV-carried sgRNAs, Ube4b and another target gene are knocked out on retina at the same time. C57BL/6 mice were purchased from Shanghai SLAC laboratory animals, Inc.

### Raising of Mice

The mice are bred and raised in the experimental animal center of Zhejiang University. All experiments were approved by the animal experimental committee of the First Affiliated Hospital of Zhejiang University School of Medicine (approval number 2019-059). The mice, which had free access to food and water, were housed in cages under a supply of positive pressure filtered air, and the bedding was changed frequently. Mice were not allowed to breed prior to or during inclusion in the in vivo experiment. During surgery, mice were anesthetized with tribromoethanol. Animals of both sexes were used.

### Method Details

### In Vivo Experimental Procedures and Reagents

### Production of AAVs

Vectors of AAV2-U6-sgPirh2, AAV2-U6-sgMDM2, AAV2-U6-sgCop1 and AAV2-U6-sgHAUSP, AAV2-U6-sgMDM4, AAV2-U6-sgMDM2, AAV2-U6-sgUbe4bAAV2-U6-sgPTEN, AAV2-CAG-Cre-WPRE, AAV9-hSyn-mChery, AAV9-CAG-PLAP, AAV9-Flex-DTR are all available from Vigene Biosciences. Vectors of AAV2-UbV.E4B, AAV2-UbiquitinWT are synthesized by Qingke Biotechnology. The sequence of the sgRNA is listed in table s3. All pAAVs were packaged by the virous center of Zhejiang University into AAVs of AAV2, AAV9 or AAV2/Retro serotype (titer >1×1013 genomic copies/ml).

### Intravitreal Injection and Optic Nerve Clamp Injury

For intravitreal injections, the inventors anesthetized the animals with tribromoethanol and then clamped the eyelid margin with arteriolar forceps to expose the conjunctiva. AAV (1-3 ul) at postnatal day 28 (P28) or Alexa-conjugated cholera toxin beta subunit (CTB-555, 1 mg/ml; 1-2 ul, Invitrogen) at postnatal day 54 (P54) was injected intravitreally using plastic tubing connected to a fine glass pipette of a Hamilton syringe. Regenerated RGC axons were traced by CTB-555 injections 2-3 days prior to euthanasia. Mice with significant ocular inflammation or atrophy were sacrificed and excluded from further experiments. Two weeks after virus injection, an orbital optic nerve clamp was performed. After the mice were anesthetized, an incision was made in the conjunctiva and the optic nerve was crushed for 5 seconds at 1-2 mm behind the optic disc using a pair of forceps with a tip width of 0.1 mm.

### Unilateral Thrombotic Stroke

The procedure for single sided thrombotic stroke was adapted from previous studies 31, 42. Briefly, the animals were mounted in a stereotactic frame with the skull exposed. To cover the sensorimotor cortex corresponding to the preferred paw, a cold light source (WeiHaiLiXin, LX-D40, 40W, 9000 mW/cm2), was positioned on an opaque template with an opening (10 mm x5 mm rectangular for rats or 2.5 mm diameter circular for mice). For rats, Rose Bengal (Rose Bengal in 20 mg/kg body weight, 20 mg/ml physiological saline) was injected intravenously at the tail, 2 minutes later, and the brain was irradiated through the skull for 15 minutes. For mice, Rose Bengal (10 mg/kg body weight, 5 mg/ml Rose Bengal in physiological saline) was injected in the tail vein and 10 minutes later, the brain was illuminated through the intact skull for 15 minutes.

### Virus Injection

Newborn Ube4bf/f mice were cryoanesthetized for 30 seconds and 3 μl of AAV9-Cre or AAV9-PLAP were injected into the right sensorimotor cortex using a 10 μL Hamilton microsyringe with a glass micropipette (68606, RWD, China) tip. After injection, mice were placed on a warm pad and returned to their mothers after returning to normal color and activity. After 6 weeks, unilateral thrombo-embolic stroke was performed in the left sensorimotor cortex. To mark CST axons by forward tracking, the inventors injected a total of 4 μl AAV9-mCherry at a rate of 80 nl min-1 to the sensory motor cortex 6 weeks after stroke, 300 nl per site at 12 sites. Two weeks after virus expression mice were heart perfused. All adeno-associated viruses used by the inventors, including AAV9-mCherry and AAV9-Cre/PLAP, were produced at the various center of Zhejiang University, with titers adjusted to 1×1013 copies per ml for injection. Three days after stroke, rats were injected with 3 μl AAV9-Ubiquitin WT/UbV.E4B and AAV9-mCherry 150 nl per minute at 18 sites. Rats were placed on soft pads on a 37° C. warming blanket until fully awake. The course of treatment for unilateral thrombotic stroke is similar to that described previously.

### CSNs to Selectively Ablate Regenerating Axons on Ischemic Side of Spinal Cord

Rats received unilateral light-induced thrombotic stroke at P70 and treatment with AAV9-ubiquitinWT/UbV.E4B at P73. 14 weeks after injury, a vertebrectomy was performed at the cervical spinal cord. AAV2/Retro-Cre (ablative) or AAV2/Retro-PLAP (control) was injected stereotactically into the neck (C5-C7) spinal cord denervated side of AAV9-UbV.E4B treated rats, for procedures see Jin et al, 2015. AAV9-Flex-DTR was then injected into the uninjured sensorimotor cortex 3 days after AAV2/Retro-Cre/PLAP injection. After 2 weeks, the animals were tested for irregular intervals of horizontal ladder walking and/or pellet grasping tasks to re-evaluate their performance of skilled limb movements. Diphtheria toxin (100 mg/kg, intravenous injection) was then administered. After 2 and 4 weeks of diphtheria toxin administration, the animals are again tested for horizontal ladder walking and/or pellet grasping tasks.

### Histology

### Tissue Preparation

The anesthetized animals were perfused transmyocardially with 4%

Paraformaldehyde (PFA). Dissected tissues were incubated overnight in 4% PFA, then cryoprotected with 15% and 30% sucrose, then embedded and flash frozen in OCT. The sucrose solution contained only 15% sucrose in PBS for the optic nerve. Typically, the optic nerve is 10 μm thick in sections, the retina is 20 μm thick in sections, and the spinal cord is 25 μm. Sections were mounted on charged microscope slides at room temperature, dried and frozen until further processing. The slides are then washed and loaded with anti-fade reagents for imaging, such as for some CTB-tracked optic nerves, or further processed for immunohistochemistry. Some retinas were fixed in PFA and dissected out completely, washed with PBS, immunostained, cut radially with scissors to flatten the tissue, and mounted for imaging.

### Immunohistofluorescent Staining

Bulk staining with Tuj1 was used to determine the number of RGCs surviving two weeks following optic nerve compression. The retinas were dissected and stained according to the previous protocol. Briefly, retinas were washed three times with 1×PBS in 96-well plates and then blocked for one hour universally in PBS containing 5% donkey serum and 0.3% Triton X-100. After incubation with Tuj1 primary antibody in PBS diluted with 3% donkey serum and 0.3% Triton X-100 for 0.5-2 days at 4° C., it was washed three times with PBS and incubated with secondary antibody for 1-2h at room temperature. After the tissues were washed with PBS, the retinas were laid flat on glass slides and images were taken under a wide field fluorescence microscope (VS 120, olympus, japan). For each retina, 12 images were taken from different areas, covering the peripheral and central areas of the retina. The number of Tuj1+RGCs is calculated by a person who is not visible to different groups.

Immunohistochemistry was performed on sections, blocked typically with 5% normal donkey serum and 0.5% Triton X-100 in PBS, incubated with primary antibody in blocking solution overnight at 4° C., washed three times with PBS, and incubated with appropriate secondary antibodies in fluorescent dye ligation at room temperature.

### Antibodies

The primary antibody used was: rabbit anti-RFP (1:500, Abcam ab34771); rabbit anti-phosphorylation S6 Ser235/236 (1:500, Cell Signaling 4857); rabbit anti-RBPMS (1:500, Abcam ab194213); mouse anti-TUJ1 (1:400, BioLegend 801213); rabbit anti-Ube4b (1:500, Invitrogen PA5-22023), mouse anti-p53 (1:500, Cell Signaling 2524S); mouse anti-HA.11 epitope tag (1:1000, BioLegend 901516); rabbit anti-Klhl22 (1:1000, ProteinTech 16214-1-AP); mouse anti-mTOR (1:5000, Cell Signaling 9964T). The fluorescent secondary antibody used, usually from Invitrogen/Thermo-Fisher Scientific or Abcam, was raised against the host species of the primary antibody, donkey, and coupled to the fluorophore of Alexa Fluor 488; Alexa Fluor 594; Cy3, or Alexa Fluor 647 (as the case may be), is typically used at a final dilution of 1:800.

### Western Blot Analysis

Mice were lightly anesthetized with isoflurane and then decapitated. Brain tissue was dissected and then homogenized in RIPA buffer containing a protease inhibitor cocktail. After centrifugation, the supernatant was used for protein quantification for BCA detection. Equal amounts of total protein were electrophoresed on SDS-polyacrylamide gels. The isolated proteins were transferred to PVDF membrane at 4° C. Blocking with 5% milk in TBST (Tris-buffered saline with Tween-20, room temperature) and incubation with primary antibody overnight at 4° C. After washing, incubation with the appropriate HRP conjugated secondary antibody was performed for 1 hour. Proteins were then detected by Western Lightning chemiluminiscence Reagent Plus (1863097, Life Technologies, Camarillo, CA) enhanced Chemiluminescence according to the manufacturer's instructions. To verify the same loading, the inventors also tested the membrane with an antibody against GAPDH (1:1000, abclonal A19056). Immunoblot density was measured using ImageJ (NIH, Bethesda, MD).

### Examination by Microscope

For some retinal sections and retinal spreads, a single fluorescence image was obtained using a wide field fluorescence microscope (VS 120, olympus, japan). For nerve and spinal cord sections, images were taken using a confocal laser scanning microscope (A1R, nikon, japan) and automatically tiled. And projects the Z-stack onto a plane. The brightness and contrast of the image are adjusted and the pseudo color is applied for display. When imaging is used for quantification, image acquisition and processing remains unchanged.

### Behaviours Science

A total of 30 adult female Sprague Dawley rats (200-250 g, 3-4 months old) were used for histology and behavior studies. According to the protocols described in previous studies, rats were trained for a single-grain grab task and an irregular step walk task. After 3-4 weeks of training, the baseline of each rat was recorded and only animals that achieved 80% success rate in the single-grain grab task and 25% error rate in the irregular step walk task were included in further experiments.

### Single Dragee Grabbing

The task of grabbing the single dragee is performed according to a previously established procedure 43. Briefly, each rat in the experiment was placed in a tight chamber (45 cm×13 cm×40 cm), passed through the broad mud in front of the chamber, and reached and grabbed a dragee (dust free precision dragee, 45 mg, bioserv) on a shelf. Throughout the training period, the rats were food restricted to maintain more than 90% of their free feed weight. During the test, 20 dragees were administered within 10 minutes. The success rate is calculated as total score/20, and the score is given according to the following rule. If the rat retrieves the dragee directly and sends it into the mouth, a score of 1 is obtained. If the rat successfully caught the dragee, but the dragee fell in the box, a score of 0.5 would be obtained. A score of 0 is given if the rat does not grab or knock the dragee off the rack. All test procedures were videotaped (60 fps) and further analyzed. For a detailed analysis of the motion component, scoring was performed according to the Eshkol-Wachmann Motion Mark (EWMN), as described in the original study 44.

### Irregular Trapezoidal Walking

The irregular stair walking task was performed as per previous reports 1, 43. Briefly, rats were induced to walk through a ladder with crossbeams spaced at unequal intervals of 1 cm to 5 cm. In each test session, rats walked through the ladder 3 times and videotaped (60 fps) for scoring. The error rate is calculated as the number of error steps/total steps, and the number of error steps includes two types. 1) Missing: when crossing the ladder, the forelimbs miss the ladder completely or touch the ladder with wrists instead of claws; 2) slipping: in traversing the ladder, the rat is placed on the ladder with several fingers rather than claws, resulting in subsequent slipping down on the ladder. The correct procedure is defined as the palm centre is correctly placed on the ladder and the fingers are closed.

### Quantification and Statistical Analysis

### Involving Quantification of Regenerating Axons

To quantify the regeneration of axons followed by fluorescent CTB after optic nerve compression, the optic nerve was dissected carefully and sectioned longitudinally by cryosectioning (slice thickness: t=10 μm) the optic nerve. The continuously collected optic nerve tissue was imaged under a confocal microscope (A1R, Nikon, Japan; 20-fold objective) with CTB channel. Optic nerve section images were derived and viewed in ImageJ. CTB+axons were counted at multiple distances (250 μm, 500 μm, 1000 μm, 1500 μm, 2000 μm, and so on) along the optic nerve, and retrograde progression from the site of the pinch was initiated. 3-4 images per optic nerve were counted. The counts were converted to the density of axons, which were then multiplied by the approximate cross-sectional area of the nerve (estimated diameter=250 μm) to estimate the total number of axons in each nerve. The estimated number of regenerating axons at different distances per optic nerve compression site was calculated using the following formula: ad=πr2*(number of axons/μm/[10 μm*(nerve width at counting site)]. The average of the estimates for each optic nerve was statistically tested as a biological sample. The measurements are made by an experimenter who keeps the conditions secret.

To quantify the number of regenerating axons, a horizontal line was first drawn through the central canal and across the lateral edges of the gray matter, and fibers were counted across the spinal cord midline under a 20-fold magnification. Four vertical lines (trunk, D1, D2, and D3) are drawn, dividing the horizontal line into three equal parts, starting from the central tube to the lateral edges. The main lines represent fibers across the centerline, while D1, D2, and D3 refer to regenerated fibers at different distances from the centerline. Only the fibers passing through these four lines were counted on each slice. Results are presented as normalized to the number of CST fibers counted at the medullary level. To determine the distribution of CST axons, images were imported in Python and analyzed, eroding all CST axons to a single pixel width. Thus, the total number of pixels of a particular region corresponds to the overall density of CST tags for the particular region in the slice. The pixilated data was further processed with python to generate a heat map, red representing the highest axon density, blue the lowest density, and white the background of the image. To analyze the correlation between regenerated CST axons and the success rate of the dragee test, the inventors plotted an average axon number index per animal.

### Involving Quantification of RGC Cell Bodies

To quantify RGCs, the eye was cryosectioned at a thickness of 20 μm. Sections were stained with anti-RBPMS (selective marker for mammalian retinal ganglion cells) to reveal RGCs. Fluorescence images were obtained on a fluorescence microscope (VS120; Olympus; Japan; 10-fold objective lens) with a 20-fold objective lens. Each retina has 3 parts quantified near the maximum diameter of the eyeball. In intact control retinas, typically 300 to 500 cells of RBPMS+ were counted per section. The cell counts were normalized to the length of the ganglion cell layer (measured for each section in OlyVIA software), and the average value for each retina was used for subsequent statistical analysis. To quantify RGCs in the whole retina, fixed whole retinas were first stained with anti-Tuj1, then cut radially into petals and observed under a fluorescence microscope (VS120; Olympus; Japan; 10X objective). Typically, six to eight regions (0.4 mm×0.4 mm each) are taken on each retina for RGCs quantification, and then averaged to generate one value per retina for subsequent statistical analysis.

### Statistical Analysis

All statistical tests were two-tailed, and the sample size n was defined as the number of individual eyes, retinas, nerves or mice in the experiment. Data were plotted and fitted using GraphPad Prism 8.0. Statistical comparisons used student's t-test, one-way analysis of variance, or two-way RM analysis of variance (Bonferroni test for post hoc comparisons). All data are presented as mean±SEM. In all cases, P<0.05 was considered statistically significant.

The specific experimental method is further described below based on the drawings.

UbV.E4B treatment in FIG. 1A-G promote regeneration of CST axons in the spinal cord and improves proficient locomotion after unilateral cortical stroke. Wherein FIG. 1A is a schematic time line of the experiment. Wild type rats received baseline behavioral training at P67, unilateral cortical subtopic thrombotic stroke at P70, unilateral cortical injection of AAV9-ubilitinWT or AAV9-UbV.E4B at P73, behavioral testing every two weeks from P77 to P155, and finally histological analysis. FIG. 1 B is schematic representation of the experimental process. 3 days after unilateral light embolism stroke, AAV9-UbiquitinWT or AAV9-UbV.E4B is injected into the cortex, and AAV9-mCherry is injected at the same time. Note lateral shoot regeneration of corticospinal axons after injury in the control (AAV9-UbiquitinWT) and experimental (AAV9-UbV.E4B) conditions. FIG. 1C is representative images of transverse sections of spinal cord (C7) stained with anti-RFP in AAV9-UbiquitinWT and AAV9-UbV.E4B treated animals. Transforming the distribution of axon regeneration to the incapacitated side into a heat map; red represents the highest number of axon pixels, blue represents the lowest, and white represents the background. FIG. 1D is quantification of crossed midline axons counted in different areas of the cervical spinal cord (C7) in AAV9-Ubiquitin WT and AAV9-UbV.E4B injection groups. The schematic diagram on the left illustrates the division of the different regions of the spinal cord. D1, D2, and D3, different lateral positions. *n=5 mice per group. Each mouse was quantified at C7 in three or four slices. FIG. 1E is performance of the single dragee grasping task. *p<0.05, repeated measures anova, Bonferroni post hoc correction, n=10 AAV9-UbiquitinWT- and n=19 AAV9-UbV.E4B-injected animals. FIG. 1F is scores of three parts in the behavioral test. ***p <0.001, AAV9-UbiquitinWT- and n=9 AAV9-UbV.E4B-injected rat n=14. g is performance of forelimbs in the task of irregular stair walking. *p<0.05, repeated measures anova, Bonferroni post hoc correction, AAV9-UbiquitinWT-animals n=9, AAV9-UbV.E4B-injected animals n=15.

FIG. 2A-C are graphs showing Ube4b as a key regulator of inhibition of central nerve axon regeneration. Wherein FIG. 2A is a time line of the experimental procedure for optic nerve regeneration. FIG. 2B representative images of optic nerve sections showing that LSL-Cas9 mice had 2 weeks post optic nerve injury, intravitreal injection of AAV2-Control-sgRNA, AAV2-Ube4b-sgRNA, AAV2-Pirh2-sgRNA, AAV2-Cop1-sgRNA, AAV2-HAUSP-sgRNA or AAV2-MDM4-sgRNA and AAV2-Cre gene knockouts, indicated CTB-labeled axons. The sites of the clamping are indicated by red asterisks. FIG. 2C quantifying the regenerated axons in (b). Data are presented as mean±SEM (n=3-9). *p <0.05, **p<0.01, ****p<0.0001 (analysis of variance and Bonferroni post test, relative to AAV 2-sgControl).

FIG. 3A-K are graphs showing that deletion of Ube4b promotes optic nerve regeneration and upregulates p53 and mTOR. Wherein FIG. 3A is a time line of the experimental procedure for optic nerve regeneration. FIG. 3B is representative images of optic nerve sections showing that Ube4bf/f mice were injected intravitreally with CTB-labeled axons of AAV2-PLAP (AAV2-control) and AAV2-Cre 2 weeks after optic nerve injury. The compressed sites are indicated by red asterisks. (a′ IN FIG. 3B) is a magnified image of the number of axons at 1000 μm from the lesion site. The scale in (FIG. 3B) and (a′) represents 100 μm. FIG. 3C is the quantification of regenerating axons at different distances from the injury site in (b) (n=4-5). Data are presented as mean±SEM. *p<0.05, ****p <0.0001 (analysis of variance and Bonferroni post). FIG. 3D is representative retinal sections stained with anti-RBPMS antibody, retinas from intact retinas or 2 weeks post-injury from prior injections of AAV2-PLAP or AAV2-Cre. e is the same as the quantification in (FIG. 3D). RBPMS positive cells of the ganglion cell layer were imaged, three sections of each retina were quantified, and lengths were counted normalised. Data are presented as mean±SEM (n=4-8). ****p<0.0001 (analysis of variance and Bonferroni post). FIG. 3F. g and h immunofluorescence analysis was performed on intact retinas or 2 weeks post AAV2-Control or AAV2-Cre injections with anti-Ube4b (FIG. 3F), p53 (FIG. 3G) and pS6 (FIG. 3H) antibodies in Ube4bf/f mouse retinal sections. FIG. 3I, J and K quantify the fluorescence intensity of the Ube4b (FIG. 3I), p53 (FIG. 3J) and pS6 (FIG. 3K) signals shown in (FIG. 3F, G and H). Three or four mice per group were quantified in at least three discrete sections in the ganglion cell layer of each retina. Data are presented as mean±SEM. By student's t-test, *p<0.05, **p<0.01.

FIG. 4A-D are graphs showing that Ube4b gene knockout regulates axon regeneration via dual pathway mediation. Wherein, a is the time course of regeneration experiments performed by conditional knockout of Ube4b and/or p53/Klhl22. *, Injury site. c is quantification of regenerating axons at different distances distal to the lesion site 2 weeks after optic nerve injury. At least three different slices per optic nerve are quantified. Data are presented as mean±SEM (n=3-5). ***p<0.001, ****p<0.0001 (analysis of variance and Bonferroni post test, relative to control). d is the scheme of Ube4b/p53 and Ube4b/mTOR signaling pathway.

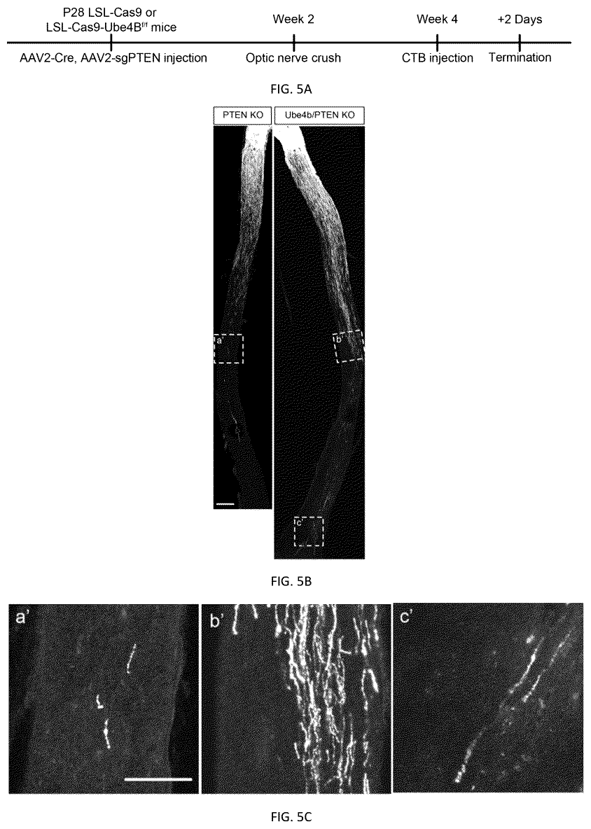

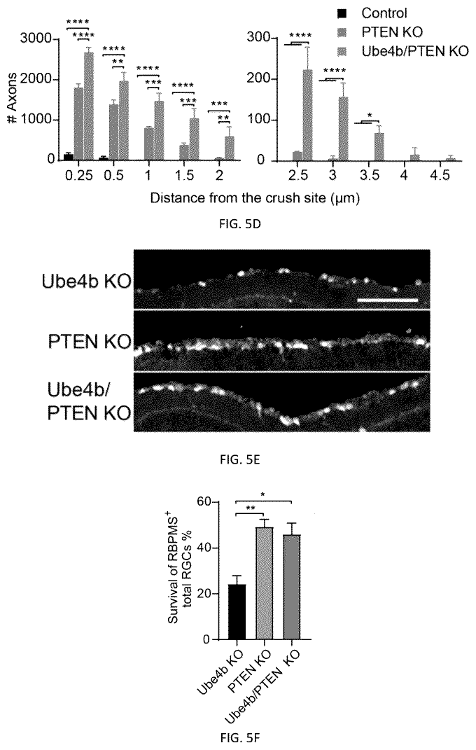

FIG. 5A-F are diagrams showing that Ube4b and PTEN double gene knockout further promote optic nerve regeneration. a is the timeline of experimental procedure for optic nerve regeneration. b is representative images, the double gene deletion of PTEN and Ube4b in RGCs induced axon regeneration rate of optic nerve 2 weeks after ONC. Red asterisks indicate the site of injury. Red arrows indicate regenerated axons of each nerve. FIG. 5C is high magnification images of the areas within the box in (FIG. 5B), which are the nerves at 2500 μm (a′, b′) and 4500 μm (c′) from the lesion. d is quantitative analysis of regenerated axons at different distances from the injury site shown in (FIG. 5B). Analysis of variance, followed by multiple comparison experiments by Bonferroni, n=3-4. *P<0.05, **P<0.01, ***P<0.001, ****P<0.0001. e is representative retinal sections stained with anti-RBPMS antibody were previously injected with AAV2-Cre and/or AAV2-sgPTEN from experimental retinas 2 weeks after injury. f is qualification of (FIG. 5E). RBPMS positive cells of the ganglion cell layer were imaged, three sections of each retina were quantified, and lengths were counted normalised. Data are presented as mean±SEM (n=3-4). *p<0.01, ***p<0.001, analysis of variance and Bonferroni post test.

FIG. 6A-F are ablations of regenerative CSNs defeats the recovery of proficient motor skills. Following stroke, rats treated with AAV9-UbV.E4B were injected intraspinally with AAV2/Retro-Cre (ablations) or AAV2/Retro-PLAP (controls) on the cervical spinal cord degeneration side (C5-C7) at P170 (1) and cortical AAV9-Flex-DTR (intact side) at P173 (2). In FIG. 6C and D, the performance of the monosaccharide pellet grasping task (FIG. 6C) and the irregular horizontal steps of forelimb integrity and denervation (FIG. 6D) were analyzed. * FIG. 6E is C7 spinal cord cross-sections of anti-RFP immunostaining represent images to indicate that CST axons from intraspinal AAV2/Retro-PLAP-(control) or

AAV2/Retro-Cre (ablation) injected animals originated from the intact side. f is quantification of midline crossing axons in the cervical spinal cord (C7) of either the intraspinal AAV2/Retro-PLAP-(control) or AAV2/Retro-Cre (ablative) injected groups. **P<0.01; ns, no statistical significance. There were 5 and 4 rats, respectively, in either AAV2/Retro-Cre-(ablative) or AAV2/Retro-PLAP (control) injected groups. Each rat was quantified in three serial sections of C7. Error bars in (FIG. 6C), (FIG. 6D), and (FIG. 6F) represent SEM.

FIG. 7A-B are graphs showing the effect of Ube4b knockout on RGC survival after injury. Representative images of FIG. 7A Tuj1 stained intact retina from intact Ube4bf/f mice injected 2 weeks after injury with AAV2-PLAP and AAV2-Cre. The scale bar represents 50 μm. FIG. 7B is Quantifying survival of RGC from (A). ANOVA test uses post test correction by Bonferroni ***p<0.001. Error bars represent SEM, at least 3 animals per group.

In FIG. 8A-B, 8A is representative retinal sections stained with anti-RBPMS antibody from different groups. The scale bar represents 100 μm. 8B quantization is as shown in (a). RBPMS positive cells in the ganglion cell layer were imaged, quantified and normalized to length counts for three sections per retina. Data are presented as mean±SEM (n=3-5). ANOVA and Bonferroni post test.

FIG. 9A-C are graphs showing acquisition of Ube4bf/f mice (control group) and Ube4b conditional knockout mice (CKO) mice. In FIG. 9B, representative western blots showed expression of Ube4b, p53, Klhl22, pS6 and mTOR in the control and Ube4b CKO mouse brains. FIG. 9C is expression of Ube4b, p53, Klhl22, pS6, and mTOR in the brain of Ube4bf/f mice (control) and Ube4b CKO mice. Data are presented as mean±SEM. *p<0.05, **p<0.01. Student's t-test. n=3 mice per group.

FIG. 10A-D are schematic diagrams of the experimental procedure. Where FIG. 10B is the timeline of the experimental procedure. Cortical injections (intact side) of AAV9-PLAP or AAV9-Cre and AAV9-mCherry from P1 Ube4bf/f mice were performed 6 weeks after unilateral thromboembolic stroke. Note that lateral shoot regeneration of corticospinal axons occurred in the spinal cord after injury under control (AAV9-PLAP) and experimental (AAV9-Cre) conditions. FIG. 10C is representative image of cervical spinal cord (C7) transected spinal cord sections, immunostained with anti-RFP to mark the CST axons as originating from the intact side (left) of cortical AAV9-PLAP-(control) or AAV9-Cre (Ube4b knockout) injected animals. The distribution of axon regeneration to the denervation side was converted into a heat map (middle); red represents the highest number of axon pixels, blue represents the lowest number, and white represents the background. Right panel in (FIG. 10C): details of boxed regions in the left panel show that RFP-labeled CST ends regenerate in the midcervical region of the marrow, Z1 or Z2 denervation region, respectively, in control or Ube4b knockout mice. Scale bar, 500 μm. In FIG. 10D, quantification of midline crossing axons in cervical spinal cord (C7) in intraspinal AAV9-PLAP-(control) or AAV9-Cre (Ube4b knockout) injected groups. Data are presented as mean±SEM. *P<0.05, **p<0.01. Student's t-test. n=5 mice per group. At least three serial sections of C7 were quantified per mouse.

FIG. 11A-D is a timeline of an experimental procedure to study optic nerve regeneration following UbV.E4B treatment. Representative images of the optic nerve sections of FIG. 11B, among others, showed that wild type mice were intravitreally injected with CTB-labeled axons of AAV2-PLAP (AAV 2-control), AAV2-Ubiquitin WT, and AAV2-UbV.E4B 2 weeks after optic nerve injury. The compressed sites are indicated by red asterisks. The scale bar represents 100 μm. FIG. 11C is quantification of regenerated axons in (FIG. 11B). Data are presented as mean±SEM (n=3-4). ****P<0.0001 (ANOVA post-assay with Bonferroni, relative to AAV2-Control). FIG. 11D is representative retinal sections stained with anti-HA antibodies from the retina 2 weeks after pre-injection of AAV2-Control, AAV2-UbiquitinWT, or AAV2-UbV.E4B lesions. The scale bar represents 50 μm.

In FIG. 12A-B, HA staining of cortical slices of animals injected with AAV9-UbV.E4B at 10 weeks of age. Asterisks indicate injection sites. Covering different parts of the entire sensorimotor cortex. The scale bar represents 1 mm.

In FIG. 13A-D, three-dimensional trajectories of representative experiments from AAV9-Ubiquitin WT- and AAV9-UbV.E4B-injected rats. Wherein the brown circles represent the location of the dragees. FIG. 13B is representative experiments with rats injected with AAV9-UbiquitinWT and AAV9-UbV.E4B three-dimensional (x, y, z) paw-closing positions relative to the dragees. The paw in the cartoon shows the direction of arrival. The circle represents the dragees with the center representing the coordinates (0, 0, 0). FIG. 13C is Hausdorff distances between the various traces were calculated in AAV9-UbiquitinWT- and AAV9-UbV.E4B-injected rats. Student's t-test. For AAV9-UbiquitinWT-injected rats, n=3, for AAV9-UbV.E4B-injected rats, n=4.

In FIG. 14A, a time chart of an experimental procedure for studying optic nerve regeneration is shown. FIG. 14B representatively shows that the absence of MDM2 and UBE4B in RGC induced faster axon regeneration in optic nerve 2 weeks after ONC. Asterisks indicate the site of pulverization. The scale bar represents 200 μm. FIG. 14C Shows the quantification of regenerating axons at different distances from the injury site shown in FIG. 14B. Analysis of variance was followed by a Bonferroni method multiple comparison test (n=3). *p<0.05, ****p<0.0001. Data are presented as SEM±mean. FIG. 14D is representative experimental retinal sections stained with anti-RBPMS antibody 2 weeks after AAV injection. The scale bar represents 50 μm. FIG. 14E is quantification of the data in FIG. 14D. RBPMS positive cells in the ganglion cell layer were imaged. Data are presented as SEM±mean (n=3). p<0.0001 (analysis of variance and post hoc test by Bonferroni).

In FIG. 16A-D is E. coli pET-His-SUMO-TAT-HA-UbV.E4B expression vector. The UbV.E4B coding sequence is inserted into an Escherichia coli protein expression vector pET-28a (+) after codon optimization (between endonuclease XolI and NdeI). FIG. 16B is PCR identification result of pET-SUMO-UbV.E4B transformant. Lanes 1, 2: PCR identification results; lane M: DNA molecular weight Marker. FIG. 16C is identifying the expression of the recombinant SUMO-UbV.E4B protein by SDS-PAGE. Lane M: protein Marker; 1. 2: the negative control induced whole cell protein distribution in the pre-supernatant and sediment; 3. 4: whole cell protein distribution in supernatants and sediments after negative control induction; 5. 6: whole-cell protein distribution in the supernatant and the sediment before the induction of E. coli BL21 (DE3)/SUMO-UbV.E4B engineered strain 1; 7. 8: whole-cell protein distribution in the supernatant and the sediment before the induction of E. coli BL21 (DE3)/SUMO-UbV.E4B engineered strain 2; 9. 10: whole cell protein distribution in the supernatant and the sediment after the induction of the engineered strain 1; 11. 12: whole cell protein distribution in the supernatant and the sediment after the induction of engineered strain 2. The rSUMO-UbV.E4B protein is indicated by a black box. FIG. 16D is the expression of E. coli BL21 (DE3)/UbV.E4B strain. Lane M: protein Marker; 1. 2: whole cell protein distribution in the supernatant and the sediment after the induction of E. coli BL21 (DE3)/UbV.E4B strain. FIG. 16E is expression of identifying recombinant UbV.E4B protein by Western blot. 1. 2: expressing HA-UbV.E4B in the supernatant and the sediment after the induction of the engineered strain 1; 3. 4: expression of rSUMO-UbV.E4B in the supernatant and the sediment after the induction of the engineered strain 2. The rSUMO-UbV.E4B protein is indicated by a black box.

FIG. 17A-B shows the results of Tricine-SDS-PAGE electrophoresis after in vitro cleavage of recombinant proteins. FIG. 17A, Tricine-SDS-PAGE electrophoresis result after rSUMO-UbV.E4B in vitro enzyme digestion. Lane 1, rSUMO-UbV.E4B without SUMO protease digestion; Lane 2, rSUMO-UBV.E4B digested by SUMO protease; Lane 3, rUBV.E4B after secondary purification and gel filtration chromatography. FIG. 17B, Tricine-SDS-PAGE electrophoresis result after rSUMO-UbiquitinWT in vitro enzyme digestion. Lane 1, rSUMO-UbiquitinWT not digested with SUMO protease; Lane 2, rSUMO-UbiquitinWT digested with SUMO protease; Lane 3, rUbiquitinWT after double purification and gel filtration chromatography.

FIG. 18A-D show that rUbV.E4B entry promotes axonal regeneration in in vitro cultured neurons. FIG. 18A shows the co-labeling of HA and Tuj1, and the scale bar is 50 μm. FIG. 18B represents axon regeneration after injury of cortical neurons cultured in vitro. The scale bar is 200 μm. FIG. 18C is the statistical result of the co-labeling rate of HA and Tuj1. All data are mean±SEM, ****p<0.0001. FIG. 18D is a statistical graph showing axon regeneration after injury of cortical neurons cultured in vitro. All data are mean±SEM, n=7, ****p<0.0001.

FIG. 19A-D are graphs showing the results of rUbV.E4B promoting optic nerve regeneration, wherein FIG. 19A shows an experimental flow chart for studying optic nerve regeneration using wild-type mice. FIG. 19B represents intravitreal injection of PBS, rUbiquitinWT or rUbV.E4B into C57BL/6 mice, and representative images of optic nerve sections 2 weeks after optic nerve injury, showing CTB-labeled axons. The lesion sites are marked with red asterisks and red arrows indicate axons that were farthest from regeneration. The scale bar is 200 microns. The right image is an enlarged image of axons at 250, 500, 1000, 1500, 2000 μm from the injury site in the dashed box of the left image. FIG. 19C, statistical map of axon regeneration. All data are mean±SEM, n=5-6, ***p<0.001, ****p<0.0001. FIG. 19D, representative images and co-labeling of immunofluorescence staining of HA, RBPMS, and DAPI in retinal sections of PBS, rUbiquitinWT, and rUbV.E4B-injected groups at 2 weeks after injury. The scale bar is 100 μm. e, Statistical graph of fluorescence intensity of HA in the RGC layer. All data are mean±SEM, n=5.

The above-mentioned embodiments are provided to further explain the objects, technical solutions and advantages of the present invention in detail, it should be understood that the above-mentioned embodiments are only examples of the present invention, and should not be construed as limiting the present invention, and any modifications, equivalent substitutions and the like made within the spirit and principle of the present invention should be included in the protection scope of the present invention.

1. A method for promoting central nervous system regeneration, comprising: (a) administrating a patient with an effective amount of a UbV.E4B protein, wherein the UbV.E4B protein promotes central nervous system axon regeneration by selectively inhibiting Ube4b ubiquitination factor, or (b) administrating a patient with an effective amount of a nucleic acid encoding the UbV.E4B protein of claim 1, wherein the UbV.E4B protein obtained by expressing the nucleic acid encoding the UbV. E4B protein selectively inhibits Ube4b ubiquitination factor, thereby promoting central nervous system axon regeneration.

2. (canceled)

3. The method according to claim 1, wherein the central nervous system regeneration is optic nerve regeneration, brain nerve axon regeneration, and/or, spinal nerve axon regeneration.

4. (canceled)

5. A method for screening a medicament promoting central nervous system axon regeneration, comprising: using a Ube4b ubiquitination factor protein as a target substance.

6. The method according to claim 5, wherein the medicament promoting central nervous system regeneration is an inhibitor of the Ube4b ubiquitination factor protein.

7. The method according to claim 5, wherein the medicament promoting central nervous system regeneration is selected from the group consisting of a protein, an antibody, a nucleic acid medicament, and a small molecule medicament.

8. The method according to claim 5, comprising: screening for a substance capable of allosterically inactivating the Ube4b ubiquitination factor protein, or screening for a substance capable of competitively occupying the natural action site of the Ube4b ubiquitination factor protein,
or screening for a substance capable of inhibiting the biosynthesis of the Ube4b ubiquitination factor protein in vivo.

9. A pharmaceutical composition for promoting central nervous system regeneration comprising a Ube4b ubiquitination factor inhibitor of the Ube4b ubiquitination factor protein of claim 5 and a pharmaceutically acceptable carrier.

10. The pharmaceutical composition according to claim 9, wherein the Ube4b ubiquitination factor inhibitor is a UbV.E4B protein or a vector comprising a nucleic acid sequence encoding a UbV.E4B protein.

11. The pharmaceutical composition according to claim 9, further comprising a transcriptional inhibitor or expression inhibitor of PTEN gene, and/or comprising a transcriptional inhibitor or expression inhibitor of MDM2 gene.

12. The pharmaceutical composition according to claim 9, further comprising a MDM2-p53 inhibitor.

13. The pharmaceutical composition according to claim 12, wherein the MDM2-p53 inhibitor is APG-115.

14. The pharmaceutical composition according to claim 9, wherein the pharmaceutical composition is formulated for administration to the brain, spinal cord, or optic nerve, or the pharmaceutical composition is formulated and administered intracerebroventricularly, intranasally, intracranially, intracerebellarly, or intrathecally.

15. A modified UbV.E4B protein linked to a membrane-penetrating peptide.

16. A gene encoding the modified UbV.E4B protein of claim 15 with a nucleotide sequence as shown in SEQ ID NO: 1.


SEQUENCE LISTING (43 sequences)

SEQ ID NO: 1 - DNA, 585 bp, synthetic construct
  source 1..585: mol_type = other DNA
atgagcgacagcgaagttaaccaggaagcgaaaccggaagtgaaaccggaagttaaaccggaaacccacattaatctgaaagttagcgacgggagcagcgaaatcttctttaaaatcaaaaagaccaccccgctgcgtcgtctgatggaagcatttgcaaaacgtcagggaaaagaaatggactcactgcgcttcctgtatgatggaattcgtattcaggcagatcagaccccggaagatctggacatggaagataatgatattattgaagcccatcgcgaacagattggaggtcgtaaaaagcgtcgtcagcgtcgtcgttatccgtatgacgttccggattatgccatgtggatttttgttcagaccctgatgcgtaacaccattaccctggaagtggaaccgagcgataccatcgaaaatgttaaagcaaaaatccaggataaagagggtattccgccggatcagcagtttctgatttttaccggtaccctgctggaagacggccgtacactgagcgattataatattcagaaagaaagtctgctgcgcctggtttggcgtccgcgtggccctctgaattaa

SEQ ID NO: 2 - DNA, 237 bp, synthetic construct
  source 1..237: mol_type = other DNA
atgtggatttttgttcagaccctgatgcgtaacaccattaccctggaagtggaaccgagcgataccatcgaaaatgttaaagcaaaaatccaggataaagagggtattccgccggatcagcagtttctgatttttaccggtaccctgctggaagacggccgtacactgagcgattataatattcagaaagaaagtctgctgcgcctggtttggcgtccgcgtggccctctgaattaa

SEQ ID NO: 3 - DNA, 231 bp, Homo sapiens
  source 1..231: mol_type = unassigned DNA
cttcagatcttcgtgaagactctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtgctccgtctcagaggtgggtga

SEQ ID NO: 4 - RNA, 20 nt, synthetic construct
  source 1..20: mol_type = other RNA
aactgacgccttcgctgctc

SEQ ID NO: 5 - RNA, 20 nt, synthetic construct
  source 1..20: mol_type = other RNA
atctctgtcgcggtccttcc

SEQ ID NO: 6 - RNA, 20 nt, synthetic construct
  source 1..20: mol_type = other RNA
ttcccgaatccacatccacc

SEQ ID NO: 7 - RNA, 20 nt, synthetic construct
  source 1..20: mol_type = other RNA
cctgtgtaggagttgcccat

SEQ ID NO: 8 - RNA, 20 nt, synthetic construct
  source 1..20: mol_type = other RNA
ctcttctgtagattcgacgg

SEQ ID NO: 9 - RNA, 20 nt, synthetic construct
  source 1..20: mol_type = other RNA
tgcctaaccacgaatcttcg

SEQ ID NO: 10 - RNA, 20 nt, synthetic construct
  source 1..20: mol_type = other RNA
gctagatcgtttcaaagtca

SEQ ID NO: 11 - RNA, 20 nt, synthetic construct
  source 1..20: mol_type = other RNA
gtgacaagctttatacctgc

SEQ ID NO: 12 - RNA, 20 nt, synthetic construct
  source 1..20: mol_type = other RNA
gaattgtccaatatgcttgg

SEQ ID NO: 13 - RNA, 20 nt, synthetic construct
  source 1..20: mol_type = other RNA
tacagtcttcacaagtctgc

SEQ ID NO: 14 - RNA, 20 nt, synthetic construct
  source 1..20: mol_type = other RNA
atacggtgctactcactgag

SEQ ID NO: 15 - RNA, 20 nt, synthetic construct
  source 1..20: mol_type = other RNA
gatggtcttaccaaaagctg

SEQ ID NO: 16 - RNA, 20 nt, synthetic construct
  source 1..20: mol_type = other RNA
aaagactcacccaagaaatt

SEQ ID NO: 17 - RNA, 20 nt, synthetic construct
  source 1..20: mol_type = other RNA
caaaatgtggccacagcttt

SEQ ID NO: 18 - RNA, 20 nt, synthetic construct
  source 1..20: mol_type = other RNA
caagtgtattcatcagagtt

SEQ ID NO: 19 - RNA, 20 nt, synthetic construct
  source 1..20: mol_type = other RNA
caaaaacttacgcaactcca

SEQ ID NO: 20 - RNA, 20 nt, synthetic construct
  source 1..20: mol_type = other RNA
agaacaagtggctgattcgc

SEQ ID NO: 21 - RNA, 20 nt, synthetic construct
  source 1..20: mol_type = other RNA
tcttcagcactgcttgtgcg

SEQ ID NO: 22 - RNA, 20 nt, synthetic construct
  source 1..20: mol_type = other RNA
tgcctgtacaaaaacttcaa

SEQ ID NO: 23 - RNA, 20 nt, synthetic construct
  source 1..20: mol_type = other RNA
agacaccagttggcgctccg

SEQ ID NO: 24 - RNA, 20 nt, synthetic construct
  source 1..20: mol_type = other RNA
tccaagtcaagaccgactga

SEQ ID NO: 25 - RNA, 20 nt, synthetic construct
  source 1..20: mol_type = other RNA
acatcagcttctattaacac

SEQ ID NO: 26 - RNA, 20 nt, synthetic construct
  source 1..20: mol_type = other RNA
atgtaggtaatgcactatct

SEQ ID NO: 27 - RNA, 20 nt, synthetic construct
  source 1..20: mol_type = other RNA
accttcagtcggtcttgact

SEQ ID NO: 28 - RNA, 20 nt, synthetic construct
  source 1..20: mol_type = other RNA
gaagcagctctatgatcaac

SEQ ID NO: 29 - RNA, 20 nt, synthetic construct
  source 1..20: mol_type = other RNA
cgacacttacactatgaaag

SEQ ID NO: 30 - RNA, 20 nt, synthetic construct
  source 1..20: mol_type = other RNA
ttgaagttgttaaagtccgt

SEQ ID NO: 31 - RNA, 20 nt, synthetic construct
  source 1..20: mol_type = other RNA
acctcttagtcataatatac

SEQ ID NO: 32 - RNA, 20 nt, synthetic construct
  source 1..20: mol_type = other RNA
tattgttcaaatgatctcct

SEQ ID NO: 33 - RNA, 20 nt, synthetic construct
  source 1..20: mol_type = other RNA
ttcagatcactcccaccttc

SEQ ID NO: 34 - RNA, 20 nt, synthetic construct
  source 1..20: mol_type = other RNA
accgccaaatttaactgcag

SEQ ID NO: 35 - RNA, 20 nt, synthetic construct
  source 1..20: mol_type = other RNA
gcagcaattcactgtaaagc

SEQ ID NO: 36 - RNA, 20 nt, synthetic construct
  source 1..20: mol_type = other RNA
tgtcatcttcacttagccat

SEQ ID NO: 37 - RNA, 20 nt, synthetic construct
  source 1..20: mol_type = other RNA
acaatattgatgatgtagta

SEQ ID NO: 38 - RNA, 20 nt, synthetic construct
  source 1..20: mol_type = other RNA
aatcccatagcaataatatt

SEQ ID NO: 39 - RNA, 20 nt, synthetic construct
  source 1..20: mol_type = other RNA
ctcattccggtggtacataa

SEQ ID NO: 40 - RNA, 20 nt, synthetic construct
  source 1..20: mol_type = other RNA
acagttgtacatctcgtcag

SEQ ID NO: 41 - RNA, 20 nt, synthetic construct
  source 1..20: mol_type = other RNA
tgagggcttaccatcaccat

SEQ ID NO: 42 - RNA, 20 nt, synthetic construct
  source 1..20: mol_type = other RNA
ataagcctgaaaatgtctcc

SEQ ID NO: 43 - RNA, 21 nt, synthetic construct
  source 1..21: mol_type = other RNA
gcgtcgtgactgggaaaaccc